(19) United States
(12) Patent Application Publication
(10) Pub. No.: US 2002/0137736 A1
(43) Pub. Date: Sep. 26, 2002

(54) Novel compounds

(76) Inventors: Kenneth Mattes, Waltham, MA (US); Robert Murray, Waltham, MA (US); Eifion D Phillips, Wilmington (DE); Hans Schmitthenner, Rochester, NY (US)

Correspondence Address: Nixon & Vanderhye, 1100 North Glebe Road 8th Floor, Arlington, VA, 22201-4714, US

(21) Appl. No.: 09/763,835

(22) PCT Filed: Dec. 14, 2000
(86) PCT No.: PCT/SE00/02539

(30) Foreign Application Priority Data
Dec. 20, 1999 (SE) 9904676-5

(51) Int. Cl. A61K 31/5377; A61K 31/496; A61K 31/4025; A61K 31/337; C07D 413/02; C 07D 4 9/02; C 07D 4 5/02

(52) U.S. Cl. 514/210.19; 514/231.2; 514/408; 514/317; 514/438; 514/471; 544/162; 544/374; 546/213; 546/214; 548/469; 548/465; 548/950; 549/74; 549/491; 514/252.13; 514/254.1

(57) ABSTRACT

There are provided novel compounds of formula (I)

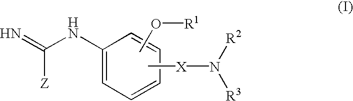

wherein R¹, R², R³, X and Z are as defined in the Specification and optical isomers, racemates and tautomers thereof and pharmaceutically acceptable salts thereof; together with processes for their preparation, compositions containing them and their use in therapy. The compounds are inhibitors of the enzyme nitric oxide synthase.

### FIELD OF THE INVENTION

This invention relates to new amidine derivatives, processes for their preparation, compositions containing them and their use in therapy.

### BACKGROUND OF THE INVENTION

Nitric oxide is produced in mammalian cells from L-arginine by the action of specific nitric oxide synthases (NOSs). These enzymes fall into two distinct classes—constitutive NOS (cNOS) and inducible NOS (iNOS). At the present time, two constitutive NOSs and one inducible NOS have been identified. Of the constitutive NOSs, an endothelial enzyme (ecNOS) is involved with smooth muscle relaxation and the regulation of blood pressure and blood flow, whereas the neuronal enzyme (ncNOS) serves as a neurotransmitter and appears to be involved in the regulation of various biological functions such as cerebral ischaemia. Inducible NOS has been implicated in the pathogenesis of inflammatory diseases. Specific regulation of these enzymes should therefore offer considerable potential in the treatment of a wide variety of disease states.

Considerable effort has been expended in efforts to identify compounds that act as specific inhibitors of one or more isoforms of the enzyme nitric oxide synthase. The use of such compounds in therapy has also been widely claimed.

WO 95/05363 discloses compounds of generic structure

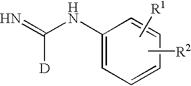

wherein D represents an aromatic ring; R¹ represents hydrogen, alkyl C1 to 6 or halogen; and R² represents a variety of nitrogen containing side-chains. The compounds have nitric oxide synthase inhibitory activity.

It has now surprisingly been found that corresponding compounds wherein R¹ represents alkoxy or a derivative thereof and which therefore are not within the generic scope of WO 95/05363 possess unexpectedly advantageous properties. Such compounds are the subject of the present application.

### DISCLOSURE OF THE INVENTION

According to the invention we provide a compound of formula (I)

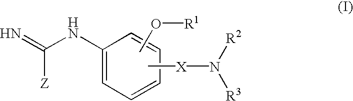

wherein

Z represents a furan or thiophene ring, optionally substituted by one or more substituents selected from halogen, trifluoromethyl, C1 to 6 alkyl, C1 to 6 alkoxy, hydroxy, amino, S(O)_{q}R⁴, CO₂R⁵ and CONR⁶R⁷;

X represents C1 to 6 alkyl;

R¹ represents hydrogen, C1 to 6 alkyl, C1 to 6 alkyl-O—R⁸, C1 to 6 alkyl-NR⁹R¹⁰ or phenyl;

said phenyl being optionally substituted by one or more substituents selected from halogen, trifluoromethyl, C1 to 6 alkyl, C1 to 6 alkoxy, hydroxy and amino;

R² and R³ independently represent hydrogen, C1 to 6 alkyl, C2 to 7 alkanoyl or —(CH₂)ₙ₋₁CH₃₋ₘFₘ;

or the group NR²R³ represents azetidinyl, pyrrolidinyl, piperidinyl, morpholinyl; or piperazinyl optionally 4-substituted by C1 to 6 alkyl;

or the groups X and R² are joined together such that the group X—N—R² represents a saturated 4 to 7 membered azacyclic ring;

R⁴, R⁵, R⁶ and R⁷ independently represent hydrogen or C1 to 6 alkyl;

R⁸ represents hydrogen, C1 to 6 alkyl or C1 to 6 alkyl substituted by hydroxy or C1 to 6 alkoxy;

R⁹ and R¹⁰ independently represent hydrogen or C1 to 6 alkyl;

or the group NR⁹R¹⁰ represents azetidinyl, pyrrolidinyl, piperidinyl, morpholinyl; or piperazinyl optionally 4-substituted by C1 to 6 alkyl;

m represents an integer 1, 2 or 3;

n represents an integer 1 to 6;

q represents an integer 0, 1 or 2;

and optical isomers, racemates and tautomers thereof and pharmaceutically acceptable salts thereof.

In one preferred embodiment, Z represents unsubstituted 2-thienyl or 3-thienyl. More preferably 2-thienyl

Preferably the substituent OR¹ in formula (I) is in the ortho or para position relative to the amidine group. More preferably the substituent OR¹ in formula (I) is in the para position relative to the amidine group, as shown in formula (IA).

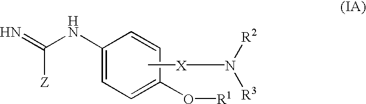

Preferably the substituent —X—NR²R³ in formula (I) is in the meta position relative to the amidine group, as shown in formula (IB).

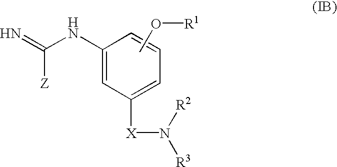

In another preferred embodiment, X represents CH₂.

Preferably R1 represents C1 to 6 alkyl. More preferably OR¹ represents methoxy or cyclopentyloxy.

In one preferred embodiment, R² and R³ independently represent hydrogen or C1 to 6 alkyl.

Particular compounds of the invention include:

N-{4-methoxy-3-[(methylamino)methyl]phenyl}-2-thiophenecarboximidamide;

N-[4-methoxy-3 -(1-pyrrolidinylmethyl)phenyl}-2-thiophenecarboximidamide;

N-[4-methoxy-3-(1-morpholinylmethyl)phenyl}-2-thiophenecarboximidamide;

N-{4-methoxy-3-[(4-methyl-1-piperazinyl)methyl]phenyl}-2-thiophenecarboximidamide,

N-{4-propoxy-3-[(methylamino)methyl]phenyl}-2-thiophenecarboximidamide;

N-{4-(cyclopentyloxy)-3-[(methylamino)methyl]phenyl}-2-thiophenecarboximidamide;

N-[4-cyclopentyloxy-3-(1-pyrrolidinylmethyl)phenyl}-2-thiophenecarboximidamide;

N-[4-methoxy-3-(1-morpholinylmethyl)phenyl}-3-thiophenecarboximidamide;

N-{2-methoxy-5-[(methylamino)methyl]phenyl}-2-thiophenecarboximidamide;

N-{2-methoxy-3-[(methylamino)methyl]phenyl}-2-thiophenecarboximidamide;

N-{4-methoxy-3-[(methylamino)methyl]phenyl}-2-furancarboximidamide;

N-(3-{[(2-fluoroethyl)amino]methyl}-4-methoxyphenyl)-2-thiophenecarboximidamide;

N-[3-(aminomethyl)-4-methoxyphenyl)-2-thiophenecarboximidamide:

N-(3-{[(2,2-difluoroethyl)amino)methyl-4-methoxyphenyl)-2-thiophenecarboximidamide;

N-(4-methoxy-3-{[(2,2,2-trifluoroethyl)amino]methyl}phenyl)-2-thiophenecarboximidamide;

N-(3-[(cyclopropylamino}methyl]-4-methoxyphenyl}-2-thiophenecarboximidamide;

N-{3-[(diethylamino)methyl]-4-methoxyphenyl}-2-thiophenecarboximidamide;

N-{3-[(isopropylamino)methyl]-4-methoxyphenyl}-2-thiophenecarboximidamide;

N-{4-isopropoxy-3-[{methylamnino}methyl]phenyl}-2-thiophenecarboximidamide;

N-{4-isopropoxy-3-[{isopropylamino}methyl]phenyll}-2-thiophenecarboximidamide;

N-[4-isopropoxy-3-(1-pyrrolidinylmethyl)phenyl]-2-thiophenecarboximidamide;

N-{4-(1-ethylpropoxy)-3-[(methylamino)methyl]phenyl}-2-thiophenecarboximidamide;

N-{4-(1-ethylpropoxy)-3-[(isopropylamino)methyl]phenyl}-2-thiophenecarboximidamide;

N-{4-cyclopentyloxy-3-[(isopropylamino)methyl]phenyl}-2-thiophenecarboximidamide;

N-{3-[(methylamino)methyl]-4-phenoxyphenyl}-2-thiophenecarboximidamide;

N-{3-[(isopropylamino)methyl]-4-phenoxyphenyl}-2-thiophenecarboximidamide;

N-{3-[(cyclopropylamino)methyl]-4-phenoxyphenyl}-2-thiophenecarboximidamide;

N-(3-{[(2-fluoroethyl)amino]methyl}-4-phenoxyphenyl)-2-thiophenecarboximidamide;

N-{4-methoxy-3-[1-(methylamino)ethyl]phenyl}-2-thiophenecarboximidamide;

N-[4-methoxy-3-(1-methyl-2-pyrrolidinyl)phenyl]-2-thiophenecarboximidamide;

N-[4-methoxy-3-(2-pyrrolidinyl)phenyl]-2-thiophenecarboximidamide; and pharmaceutically acceptable salts thereof.

In one aspect the invention includes compounds of formula (I)

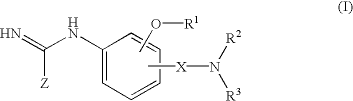

wherein

Z represents a furan or thiophene ring, optionally substituted by halogen, trifluoromethyl,

C1 to 6 alkyl or C1 to 6 alkoxy;

X represents C1 to 6 alkyl;

R¹ represents hydrogen, C1 to 6 alkyl, C1 to 6 alkyl-O—R⁸ or C1 to 6 alkyl-NR⁹R¹⁰;

R² and R³ independently represent hydrogen, C1 to 6 alkyl, C2 to 7 alkanoyl or -(CH₂)ₙF;

or the group NR²R³ represents azetidinyl, pyrrolidinyl, piperidinyl, morpholinyl; or piperazinyl optionally 4-substituted by C1 to 6 alkyl;

R⁸ represents hydrogen, C1 to 6 alkyl or C1 to 6 alkyl substituted by hydroxy or C1 to 6 alkoxy;

R⁹ and R¹⁰ independently represent hydrogen or C1 to 6 alkyl;

or the group NR⁹R¹⁰ represents azetidinyl, pyrrolidinyl, piperidinyl, morpholinyl; or piperazinyl optionally 4-substituted by C1 to 6 alkyl;

n represents an integer 1 to 6;

and optical isomers, racemates and tautomers thereof and pharmaceutically acceptable salts thereof.

Unless otherwise indicated, the term “C1 to 6 alkyl” referred to herein denotes a straight or branched chain alkyl group having from 1 to 6 carbon atoms and/or a cyclic alkyl group having from 3 to 6 carbon atoms. Examples of such groups include methyl, ethyl, n-propyl, i-propyl, n-butyl, i-butyl, t-butyl, cyclopropyl, cyclopropylmethyl, cyclopentyl, methylcyclopentyl, cyclopentylmethyl and cyclohexyl.

Unless otherwise indicated, the term “C2 to 7 alkanoyl” referred to herein denotes a straight or branched chain alkyl group having from 1 to 6 carbon atoms or a cyclic alkyl group having from 3 to 6 carbon atoms bonded to a carbonyl (CO) group. Examples of such groups include acetyl, propionyl, iso-butyryl, valeryl, pivaloyl, cyclopentanoyl and cyclohexanoyl.

Unless otherwise indicated, the term “C1 to 6 alkoxy” referred to herein denotes an oxygen substituent bonded to a straight or branched chain alkyl group having from 1 to 6 carbon atoms and/or a cyclic alkyl group having from 3 to 6 carbon atoms. Examples of such groups include methoxy, ethoxy, n-propoxy, i-propoxy, n-butoxy, i-butoxy, s-butoxy, t-butoxy, cyclopropyloxy, cyclopropylmethoxy, cyclopentyloxy, methylcyclopentyloxy. cyclopentylmethoxy and cyclohexyloxy.

The term “C1 to 6 alkyl-O—R⁸” denotes a C1 to 6 alkyl group, as defined above, in which one hydrogen atom is replaced by a group O—R⁸. The term “C1 to 6 alkyl-NR⁹R¹⁰” is to be interpreted analogously.

Unless otherwise indicated, the term “halogen” referred to herein denotes fluorine, chlorine, bromine and iodine.

Examples of compounds wherein the groups X and R² are joined together such that the group X—N—R² represents a saturated 4 to 7 membered azacyclic ring include compounds such as those of formulae (IC) and (ID)

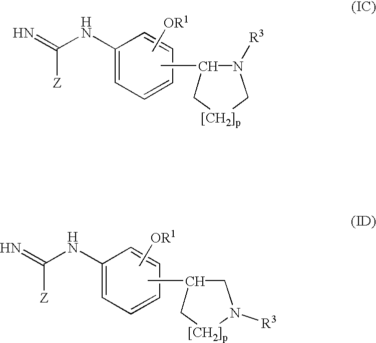

wherein p represents an integer 0 to 3.

The present invention includes compounds of formula (I) in the form of salts, in particular acid addition salts. Suitable salts include those formed with both organic and inorganic acids. Such acid addition salts will normally be pharmaceutically acceptable although salts of non-pharmaceutically acceptable acids may be of utility in the preparation and purification of the compound in question. Thus, preferred salts include those formed from hydrochloric, hydrobromic, sulphuric, phosphoric, citric, tartaric, lactic, pyruvic, acetic, succinic, fumaric, maleic, methanesulphonic and benzenesulphonic acids.

According to the invention, we further provide a process for the preparation of compounds of formula (I), and optical isomers and racemates thereof and pharmaceutically acceptable salts thereof, which comprises preparing a compound of formula (I) by:

(a) reacting a corresponding compound of formula (II) or a salt thereof

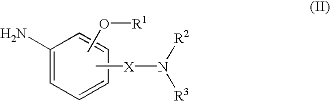

wherein R¹, R², R³ and X are as defined above, with a compound of formula (III) or a salt thereof

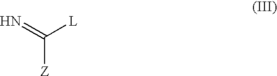

wherein Z is as defined above and L represents a leaving group; or

(b) reacting a corresponding compound of formula (IV) or a salt thereof

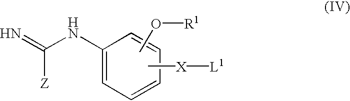

wherein R¹, X and Z are as defined above and L¹ is a leaving group, with a compound of formula HNR²R³ or a salt thereof, wherein R² and R³ are as defined above; or

(c) preparing a compound of formula (I) wherein X represents -CH₂-by reduction of a corresponding compound wherein X represents-CO-(formula V)

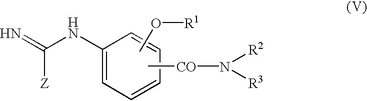

and where desired or necessary converting the resultant compound of formula (I), or another salt thereof, into a pharmaceutically acceptable salt thereof, or vice versa, and where desired converting the resultant compound of formula (I) into an optical isomer thereof.

In process (a), the reaction will take place on stirring a mixture of the reactants in a suitable solvent, for example a lower alkanol such as ethanol, 2-propanol or tert-butanol, at a temperature between room temperature and the reflux temperature of the solvent. The reaction may optionally be carried out under an atmosphere of an inert gas such as nitrogen or argon. The reaction time will depend inter alia on the solvent and the nature of the leaving group, and may be up to 48 hours; however it will typically be from 1 to 5 hours. Suitable leaving groups L include thioalkyl, sulfonate, trifluoromethylsulfonate, halide, alkoxide, aryloxide and tosylate groups; others are recited in “Advanced Organic Chemistry”, J. March (1985) 3^{rd} Edition on page 315 and are well known in the art. We find thioalkyl, especially thiomethyl or thioethyl, to be particularly useful.

In process (b), the amination reaction is performed by reacting a compound of formula (IV) with an amine in an inert solvent. Suitable leaving groups include sulfonate, trifluorosulfonate, tosylate, and halides selected from the group chloride, bromide or iodide. The nucleophile can be a primary or secondary amine in the presence of a base. This base can be either an excess of the amine nucleophile or can be an additive to the reaction mixture. Potential basic additives are metal carbonate, especially alkali metal carbonates, metal oxides and hydroxides, and tertiary amine bases such as diisopropylethylamine. Suitable organic solvents are those such as acetonitrile, dioxane, N,N-dimethylforrnamide, N-methyl-2-pyrrolidinone, tetrahydrofuran, dimethylsulfoxide, sulfolane and C1 to 4 alcohols. In a preferred embodiment, the leaving group is chloride.

Salts of compounds of formula (I) may be formed by reacting the free base or a salt, enantiomer, tautomer or protected derivative thereof, with one or more equivalents of the appropriate acid. The reaction may be carried out in a solvent or medium in which the salt is insoluble, or in a solvent in which the salt is soluble followed by subsequent removal of the solvent in vacuo or by freeze drying,. Suitable solvents include, for example, water, dioxan, ethanol, 2-propanol, tetrahydrofuran or diethyl ether, or mixtures thereof. The reaction may be a metathetical process or it may be carried out on an ion exchange resin.

Certain novel intermediates of formulae (II), (IV) and (V) form another aspect of the invention.

Compounds of formula (II) may be prepared by methods that will be generally apparent to the man skilled in the art. In particular, these methods include the reduction of a corresponding compound of formula (VI)

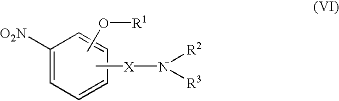

wherein R¹, R², R³ and X are as defined above.

Such reductions may be achieved using various methods that are well known in the art.

Compounds of formula (III) are either known or may be prepared by known methods. For example, compounds of formula (III) in which L represents thioalkyl may be prepared by treatment of the corresponding thioamide of formula (VII)

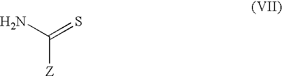

wherein Z is as defined above; with an alkyliodide.

Compounds of formula (VI) may be prepared by methods that will be generally apparent to the man skilled in the art. Such methods include:

(a) reaction of a compound of formula (VIII)

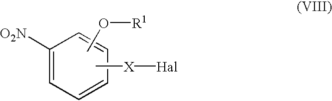

wherein R¹ and X are as defined above and Hal represents a halogen, with an amine of formula HNR²R³ wherein R² and R³ are as defined above;

(b) when X represents-CO-, reacting a compound of formula (IX)

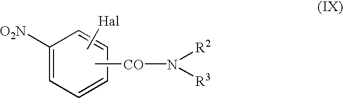

wherein R² and R³ are as defined above and Hal represents a halogen, with an metal alkoxide, M-OR¹, wherein R¹ is as defined above and M represents a metal, particularly an alkali or alkaline earth metal such as sodium or potassium; and

(c) by reductive amination of a compound of formula (X)

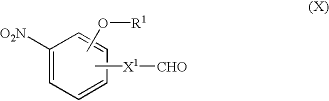

wherein X¹ represents an alkyl group having one less CH₂ group than X, and R¹ and X are as defined above; with an amine of formula HNR²R³.

Compounds of formula (IV) may be prepared from corresponding compounds of formula (XI)

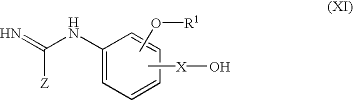

wherein R¹, X and Z are as defined above, using methods that are generally well known in the art.

Compounds of formulae (VI), (VII), (VIII), (IX), (X) and (XI) are either known or may be prepared by conventional methods that will be known per se to the man skilled in the art.

Intermediate compounds may be prepared as such or in protected form. In particular amine and hydroxy groups may be protected. Suitable protecting groups are described in the standard text “Protective Groups in Organic Synthesis”, 2nd Edition (1991) by Greene and Wuts. Amine protecting groups which may be mentioned include alkyloxycarbonyl such as t-butyloxycarbonyl, phenylalkyloxycarbonyl such as benzyloxycarbonyl, or trifluoroacetate. Deprotection will normally take place on treatment with aqueous base or aqueous acid.

The compounds of the invention and intermediates may be isolated from their reaction mixtures, and if necessary further purified, by using standard techniques.

The compounds of formula (I) may exist in tautomeric, enantiomeric or diastereoisomeric forms, all of which are included within the scope of the invention. The various optical isomers may be isolated by separation of a racemic mixture of the compounds using conventional techniques, for example, fractional crystallisation or HPLC. Alternatively, the individual enantiomers may be made by reaction of the appropriate optically active starting materials under reaction conditions that will not cause racemisation.

Intermediate compounds may also exist in enantiomeric forms and may be used as purified enantiomers, diastereomers, racemates or mixtures.

The compounds of formula (I), and their pharmaceutically acceptable salts, enantiomers, racemates and tautomers, are useful because they possess pharmacological activity in animals. In particular, the compounds are active as inhibitors of the enzyme nitric oxide synthase and as such are predicted to be useful in therapy. More particularly, they are in general selective inhibitors of the neuronal isoform of the enzyme nitric oxide synthase.

The compounds and their pharmaceutically acceptable salts, enantiomers, racemates and tautomers are indicated for use in the treatment or prophylaxis of diseases or conditions in which synthesis or oversynthesis of nitric oxide synthase forms a contributory part.

Examples of such diseases or conditions include hypoxia, such as in cases of cardiac arrest, stroke and neonatal hypoxia, neurodegenerative conditions including nerve degeneration and/or nerve necrosis in disorders such as ischaemia, hypoxia, hypoglycemia, epilepsy, and in external wounds (such as spinal cord and head injury), hyperbaric oxygen convulsions and toxicity, dementia, for example, pre-senile dementia, Alzheimer's disease and AIDS-related dementia, Sydenham's chorea, Parkinson's disease, Huntington's disease, multiple sclerosis, Amyotrophic Lateral Sclerosis, Korsakoffs disease, imbecility relating to a cerebral vessel disorder, sleeping disorders, schizophrenia, anxiety, depression, seasonal affective disorder, jet-lag, depression or other symptoms associated with Premenstrual Syndrome (PMS), anxiety and septic shock.

The compounds of formula (I) are also useful in the treatment and alleviation of acute or persistent inflammatory or neuropathic pain, or pain of central orhgin.

The compounds of formula (I) may also be useful in the treatment or prophylaxis of inflammation. Conditions that may be specifically mentioned include osteoarthritis, rheumatoid arthritis, rheumatoid spondylitis, gouty arthtis and other arthritic conditions, inflamed joints; eczema, psoriasis, dermatitis or other inflammatory skin conditions such as sunburn; inflammatory eye conditions including uveitis and conjunctivitis; lung disorders in which inflammation is involved, for example, asthma, bronchitis, chronic obstructive pulmonary disease, pigeon fancier's disease, farmer's lung, acute respiratory distress syndrome; bacteraemia, endotoxaemia (septic shock), aphthous ulcers, gingivitis, pyresis, pain and pancreatitis; conditions of the gastrointestinal tract including inflammatory bowel disease, Crohn's disease, atrophic gastritis, gastritis varialoforme, ulcerative colitis, coeliac disease, regional ileitis, peptic ulceration, irritable bowel syndrome, damage to the gastrointestinal tract resulting from infections by, for example, Helicobacter pylori, or from treatments with non-steroidal anti-inflammatory drugs; and other conditions associated with inflammation.

The compounds of formula (I) and their pharmaceutically acceptable salts, enantiomers, racemates and tautomers may also be useful in the treatment or prophylaxis of diseases or conditions in addition to those mentioned above. For example, the compounds may be useful in the treatment of atherosclerosis, cystic fibrosis, hypotension associated with septic and/or toxic shock, in the treatment of dysfunction of the immune system, as an adjuvant to short-term immunosuppression in organ transplant therapy, in the treatment of vascular complications associated with diabetes and in cotherapy with cytokines, for example TNF or interleukins.

Compounds of formula (I) are also predicted to show activity in the prevention and reversal of tolerance to opiates and diazepines, treatment of drug addiction and treatment of migraine and other vascular headaches. The compounds of the present invention may also show useful immunosuppressive activity, and be useful in the treatment of gastrointestinal motility disorders, in the induction of labour, and in male contraception.

The compounds may also be useful in the treatment of cancers that express nitric oxide synthase.

Compounds of formula (I) are predicted to be particularly useful in the treatment or prophylaxis of hypoxia or stroke or ischaemia or neurodegenerative conditions or schizophrenia or migraine or for the treatment of pain and especially in the treatment or prophylaxis of hypoxia or stroke or ischaemia or neurodegenerative disorders or schizophrenia or pain. We are particularly interested in the conditions selected from the group consisting of hypoxia, ischaemia, stroke, pain, anxiety, schizophrenia, Parkinson's disease, Huntington's disease and migraine and other vascular headaches.

For the treatment of Parkinson's disease, the compounds of formula (I) are expected to be particularly useful either alone, or in combination with other agents such as L-Dopa.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

Thus according to a further aspect of the invention we provide a compound of formula (I), or an optical isomer or racemate thereof or a pharmaceutically acceptable salt thereof, for use as a medicament.

According to another feature of the invention we provide the use of a compound of formula (I) or an optical isomer or racemate thereof or a pharmaceutically acceptable salt thereof, in the manufacture of a medicament for the treatment or prophylaxis of the aforementioned diseases or conditions; and a method of treatment or prophylaxis of one of the aforementioned diseases or conditions which comprises administering a therapeutically effective amount of a compound of formula (I), or an optical isomer or racemate thereof or a pharmaceutically acceptable salt thereof, to a person suffering from or susceptible to such a disease or condition.

For the above mentioned therapeutic indications, the dosage administered will, of course, vary with the compound employed, the mode of administration and the treatment desired. However, in general, satisfactory results are obtained when the compounds are administered to a human at a daily dosage of between 0.5 mg and 2000 mg (measured as the active ingredient) per day, particularly at a daily dosage of between 2 mg and 500 mg.

The compounds of formula (I), and optical isomers and racemates thereof and pharmaceutically acceptable salts thereof, may be used on their own, or in the form of appropriate medicinal formulations. Administration may be by, but is not limited to, enteral (including oral, sublingual or rectal), intranasal, or topical or other parenteral routes. Conventional procedures for the selection and preparation of suitable pharmaceutical formulations are described in, for example, “Pharmaceuticals—The Science of Dosage Form Designs”, M. E. Aulton, Churchill Livingstone, 1988.

According to the invention, there is provided a pharmaceutical formulation comprising preferably less than 95% by weight and more preferably less than 50% by weight of a compound of formula (I), or an optical isomer or racemate thereof or a pharmaceutically acceptable salt thereof, in admixture with a pharmaceutically acceptable diluent or carrier. The formulation may optionally also contain a second pharmacologically active ingredient such as L-Dopa.

The compounds of formula (I), and pharmaceutically acceptable derivatives thereof, may also be advantageously used in combination with a COX-2 inhibitor. Particularly preferred COX-2 inhibitors are Celecoxib and MK-966. The NOS inhibitor and the COX-2 inhibitor may either be formulated together within the same pharmaceutical composition for administration in a single dosage unit, or each component may be individually formulated such that separate dosages may be administered either simultaneously or sequentially.

We also provide a method of preparation of such pharmaceutical formulations which comprises mixing the ingredients.

Examples of such diluents and carriers are: for tablets and dragees: lactose, starch, talc, stearic acid; for capsules: tartaric acid or lactose; for injectable solutions: water, alcohols, glycerin, vegetable oils; for suppositories: natural or hardened oils or waxes.

Compositions in a form suitable for oral, that is oesophageal, administration include: tablets, capsules and dragees; sustained release compositions include those in which the active ingredient is bound to an ion exchange resin which is optionally coated with a diffusion barrier to modify the release properties of the resin.

The enzyme nitric oxide synthase has a number of isoforms and compounds of formula (I), and optical isomers and racemates thereof and pharmaceutically acceptable salts thereof, may be screened for nitric oxide synthase inhibiting activity by following procedures based on those of Bredt and Snyder in Proc. Nat. Acad. Sci., 1990, 87, 682-685. Nitric oxide synthase converts ³H-L-arginine into ³H-L-citrulline which can be separated by cation exchange chromatography and quantified by scintillation counting.

Screen for Neuronal Nitric Oxide Synthase Inhibiting Activity

The enzyme is isolated from rat hippocampus or cerebellum. The cerebellum or hippocampus of a male Sprague-Dawley rat (250-275 g) is removed following CO₂ anaesthesia of the animal and decapitation. Cerebellar or hippocampal supernatant is prepared by homogenisation in 50 mM Tris-HCl with 1 mM EDTA buffer (pH 7.2 at 25° C.) and centifugation for 15 minutes at 20,000 g. Residual L-arginine is removed from the supernatant by chromatography through Dowex AG-50W-X8 sodium form and hydrogen form columns successively, and further centrifugation at 1000 g for 30 seconds. For the assay, 25 μl of the final supernatant is added to each of 96 wells (of a 96 well filter plate) containing either 25 μl of an assay buffer (50 mM HEPES, 1 mM EDTA, 1.5 mM CaCl₂, pH 7.4) or 25 μl of test compound in the buffer at 22° C. and 25 μl of complete assay buffer (50 mM HEPES, 1 mM EDTA, 1.5 mM CaCl₂, 1 mM DTT, 100 μM NADPH, 10 μg/ml calmodulin, pH 7.4). Following a 10 minute equilibration period, 25 μl of an L-arginine solution (of concentration 18 μM ¹H-L-arinine, 96 nM ³H-L-arginine) is added to each well to initiate the reaction. The reaction is stopped after 10 minutes by addition of 200 μl of a slurry of termination buffer (20 mM HEPES, 2 mM EDTA, pH 5.5) and Dowex AG-50W—X8 200-400 mesh.

Labelled L-citrulline is separated from labelled L-arginine by filtering each filter plate and 75 μl of each terminated reaction is added to 3 ml of scintillation cocktail. The L-citrulline is then quantified by scintillation counting.

In a typical experiment using the cerebellar supernatant, basal activity is increased by 20,000 dpm/ml of sample above a reagent blank that has an activity of 7,000 dpm/ml. A reference standard, N-nitro-L-arginine, which gives 80% inhibition of nitric oxide synthase at a concentration of 1 μM, is tested in the assay to verify the procedure.

Screen for Human Neuronal Nitric Oxide Synthase Inhibiting Activity

Enzyme was isolated from human hippocampus, cortex or cerebellum. Cerebellar, cortical or hippocampal supernatant is prepared by homogenisation of frozen human tissue (1 to 5 g) in 50 mM Tris-HCl with 1 mM EDTA buffer (pH 7.2 at 25° C.) and centrifugation for 15 minutes at 20,000 g. Residual L-arginine is removed from the supernatant by chromatography through Dowex AG-50W—X8 sodium form and hydrogen form columns successively and further centrifugation at 1000 g for 30 seconds. Subsequently, the supernatant is passed through 2′-5′ ADP Sepharose and the human nNOS eluted with NADPH.

For the assay, 25 μl of the final supernatant is added to each of 96 wells (of a 96 well filter plate) containing either 25 μl of an assay buffer (50 mM HEPES, 1 mM EDTA, 1.5 mM CaCl₂, pH 7.4) or 25 μl of test compound in the buffer at 22° C. and 25 μl of complete assay buffer (50 mM HEPES, 1 mM EDTA, 1.5 mM CaCl₂, 1 mM DTT, 100 μM NADPH, 10 μg/ml calmodulin, pH 7.4). Following a 30 minute equilibration period, 25 μl of an L-arginine solution (of concentration 12 μM ¹H-L-arginine, 96 nM ³H-L-arginine) is added to each test tube to initiate the reaction. The reaction is stopped after 30 minutes by addition of 200 μl of a slurry of termination buffer (20 mM HEPES, 2 mM EDTA, pH 5.5) and Dowex AG-50W—X8 200-400 mesh.

Labelled L-citrulline is separated from labelled L-arginine by filtering each filter plate and 75 μl of each terminated reaction is added to 3 ml of scintillation cocktail. The L-citrulline is then quantified by scintillation counting.

In a typical experiment using the cerebellar supernatant, basal activity is increased by 20,000 dpm/ml of sample above a reagent blank that has an activity of 7,000 dpm/ml. A reference standard, N-nitro-L-arginine, which gives 80% inhibition of nitric oxide synthase at a concentration of 1 μM, is tested in the assay to verify the procedure.

Screen for Human Inducible Nitric Oxide Synthase Inhibiting Activity

Partially purified iNOS was prepared from cultured and lysed human DLD1 cells which had been activated with TNF-alpha, interferon gamma, and LPS. Centrifugation at 1000 g removed cellular debris and residual L-arginine was removed from the supernatant by chromatography through Dowex AG-50W—X8 sodium form and hydrogen form columns successively.

For the assay, 25 μl of the final supernatant is added to each of 96 wells (of a 96 well filter plate) containing either 25 μl of an assay buffer (50 rnM HEPES, 1 mM EDTA, 1.5 mM CaCl₂, pH 7.4) or 25 μl of test compound in the buffer at 22° C. and 25 μl of complete assay buffer (50 mM HEPES, 1 mM EDTA, 1.5 mM CaCl², 1 mM DTT, 100 μM NADPH, 10 μg/ml calmodulin, pH 7.4). Following a 30 minute equilibration period, 25 1i of an L-arginine solution (of concentration 12 μM ¹H-L-arginine, 96 nM ³H-L-arginine) is added to each test tube to initiate the reaction. The reaction is stopped after 30 minutes by addition of 200 μl of a slurry of termination buffer (20 mM HEPES, 2 mM EDTA, pH 5.5) and Dowex AG-50W—X8 200-400 mesh.

Labelled L-citrulline is separated from labelled L-arginine by filtering each filter plate and 75 μl of each terminated reaction is added to 3 ml of scintillation cocktail. The L-citrulline is then quantified by scintillation counting.

In a typical experiment using the DLD1 supernatant, basal activity is increased by 10,000 dpm/ml of sample above a reagent blank that has an activity of 5,000 dpm/ml. A reference standard, N-methyl-L-arginine, which gives 80% inhibition of nitric oxide synthase at a concentration of 1 μM, is tested in the assay to verify the procedure.

Screen for Endothelial Nitric Oxide Synthase Inhibiting Activity

The enzyme is isolated from human umbilical vein endothelial cells (HUVECs) by a procedure based on that of Pollock et al in Proc. Natl. Acad. Sci., 1991, 88, 10480-10484. HUVECs were purchased from Clonetics Corp (San Diego, Calif., USA) and cultured to confluency. Cells can be maintained to passage 35-40 without significant loss of yield of nitric oxide synthase. When cells reach confluency, they are resuspended in Dulbecco's phosphate buffered saline, centrifuged at 800 rpm for 10 minutes, and the cell pellet is then homogenised in ice-cold 50 mM Tris-HCl, 1 mM EDTA, 10% glycerol, 1 mM phenylmethylsulphonylfluoride, 2 μM leupeptin at pH 4.2. Following centrifugation at 34,000 rpm for 60 minutes, the pellet is solubilised in the homogenisation buffer which also contains 20 mM CHAPS. After a 30 minute incubation on ice, the suspension is centrifuged at 34,000 rpm for 30 minutes. The resulting supernatant is stored at −80° C. until use.

For the assay, 25 μl of the final supernatant is added to each of 12 test tubes containing 25 μl L-arginine solution (of concentration 12 μM ¹H-L-arginine, 64 nM ³H-L-arginine) and either 25 μl of an assay buffer (50 mM HEPES, 1 mM EDTA, 1.5 mM CaCl₂, pH 7.4) or 25 μl of test compound in the buffer at 22° C. To each test tube was added 25 μl of complete assay buffer (50 mM HEPES, 1 mM EDTA, 1.5 mM CaCl₂, 1 mM DTT, 100 μM NADPH, 10 μg/ml calmodulin, 12 μM tetrahydrobiopterin, pH 7.4) to initiate the reaction and the reaction is stopped after 10 minutes by addition of 2 ml of a termination buffer (20 mM HEPES, 2 mM EDTA, pH 5.5).

Labelled L-citrulline is separated from labelled L-arginine by chromatography over a Dowex AG-50W-X8 200-400 mesh column. A 1 ml portion of each terminated reaction mixture is added to an individual 1 ml column and the eluent combined with that from two 1 ml distilled water washes and 16 ml of scintillation cocktail. The L-citrulline is then quantified by scintillation counting.

In a typical experiment, basal activity is increased by 5,000 dpm/ml of sample above a reagent blank that has an activity of 1500 dpm/ml. A reference standard, N-nitro-L-arginine, which gives 70-90% inhibition of nitric oxide synthetase at a concentration of 1 μM, is tested in the assay to verify the procedure.

In the screens for nitric oxide synthase inhibition activity, compound activity is expressed as IC₅₀ (the concentration of drug substance which gives 50% enzyme inhibition in the assay). IC₅₀ values for test compounds were initially estimated from the inhibiting activity of 1, 10 and 100 μM solutions of the compounds. Compounds that inhibited the enzyme by at least 50% at 10 μM were re-tested using more appropriate concentrations so that an IC₅₀ could be determined.

When tested in the above screens, the compounds of Examples 1 to 31 below showed IC₅₀ values for inhibition of neuronal nitric oxide synthase of less than 10 μM and good selectivity compared to inhibition of the endothelial isoform of the enzyme, indicating that they are predicted to show particularly useful therapeutic activity.

The invention is illustrated but in no way limited by the following examples:

### PREPARATION 1

2-Thiophenecarboximidothioic acid ethyl ester hydrochloride

To a stirred solution of ethanethiol (28.4 g, 450 mmol) in dichloromethane (500 mL) at 10° C. under nitrogen was added 2-thiophenecarbonitrile (50.0 g, 450 mmol). The solution was treated with a slow stream of hydrogen chloride gas for 6 h. The reaction mixture was then allowed to warm to room temperature. After 18 h diethyl ether (200 mL) was added and a white solid crystallized out. The solid 2-thiophenecarboximidothioic acid ethyl ester hydrochloride was collected by filtration and air dried (65.8 g, 83%); m.p. 196-197° C.

### PREPARATION 2

2-Furancarboximidothioic acid ethyl ester hydrochloride

Following the procedure described in Preparation 1 but substituting 2-farancarbonitrile for 2-thiophenecarbonitrile, the title compound was prepared as a white solid in 23% yield; MS:^{m}/z 156 [M+H]⁺.

### PREPARATION 3

2-Methoxy-5-nitrobenzaldehyde

To triphenylphosphine (11.9 g, 45 mmol) in tetrahydrofuran (100 ml) was added 2-hydroxy-5-nitrobenzaldehyde (6.3 g, 38 mmol) and methanol (1.8 g, 57 mmol) followed by diethyl azodicarboxylate (7.9 g, 45 mmol) and the reaction mixture was let stir at room temperature for 1 h. The solvent was evaporated off, the residue was dissolved in ethyl acetate (50 mL) and filtered through a plug of silica. The filtrate was concentrated and chromatographed on silica gel using a gradient of 20-50% ethyl acetate in methanol to afford the title compound (4.9 g, 71.6%) as an oil; MS:^{m}/z 182 [M+H]⁺.

### PREPARATION 4

2-Methoxy-3-nitrobenzaldehyde

Following the procedure described in Preparation 3 but substituting 2-hydroxy-3-nitrobenzaldehyde for 2-hydroxy-5-nitrobenzaldehyde, the title compound was obtained as a yellow oil in 54% yield; MS:^{m}/z 182 [M+H]⁺.

### PREPARATION 5

4-Methoxy-3-nitrobenzaldehyde

Following the procedure described in Preparation 3 but substituting 4-hydroxy-3-nitrobenzaldehyde for 2-hydroxy-5-nitrobenzaldehyde, the title compound was obtained as a yellow oil in 40% yield; MS:^{m}/z 182 [M+H]⁺.

### PREPARATION 6

2-Chloro N-methyl-5-nitrobenzamide

To a solution of 2-chloro-5-nitrobenzoyl chloride (20 g, 91 mmol) in dichloromethane (200 mL) cooled to 0° C. was added an ice-cold solution of dichloromethane (100 mL) containing methylamine (11 g, 360 mmol). The reaction mixture was stirred in the cold for 1 h, diluted with dichloromethane (500 mL), and the dichloromethane solution washed sequentially with water (200 mL), 5% aqueous hydrochloric acid (150 mL), water (200 mL) and aqueous saturated sodium chloride solution (2×150 mL). The dichloromethane solution was then dried over magnesium sulphate and the solvent evaporated to afford the title compound (15.5 g, 80%) as a colourless solid; MS:^{m}/z 215 [M+H]⁺.

### PREPARATION 7

N-[3-(Chloromethyl)-4-methoxyphenyl]-2-thiophenecarboximidamide Hydrochloride

a) Methyl 2-Methoxy-5-nitrobenzoate

To methyl 2-methoxybenzoate (5.0 g, 30 mmol) dissolved in cold (0° C.) sulphuric acid (25 mL) was added portionwise potassium nitrate (3.0 g, 30 mmol) and the mixture was allowed to stir at 0° C. for 1 h. The reaction mixture was poured onto 200 mL of ice/water mixture and the solid was collected, washed well with water and air-dried; yield 3.5 g (58%); MS:^{m}/z 212 [M+H]⁺.

b) Methyl 5-Amino-2-methoxybenzoate Hydrochloride

To a Parr pressure bottle charged with methyl 2-methoxy-5-nitrobenzoate (3.5 g, 17.4 mmol) in ethanol (200 mL) was added an ethanol solution saturated with hydrogen chloride (20 mL) followed by 10% Pd/C (200 mg) and the mixture was hydrogenated at 45 psi for 1 h. The catalyst was filtered off and the solvent evaporated to afford the title compound (3.6 g, 100%) as a colourless solid; MS:^{m}/z 182 [M+H]⁺.

c) Methyl 5-{Imino(2-thienyl)methyl]amino}-2-methoxybenzoate

To methyl 5-amino-2-methoxybenzoate hydrochloride (45.6 g, 210 mmol) dissolved in ethanol (250 mL) was added pyridine (16.7 g, 210 mmol) followed by 2-thiophenecarboximidothioic acid ethyl ester hydrochloride (50.2 g, 240 mmol) and the reaction mixture was heated at 60° C. under nitrogen for 18 h. The reaction mixture was poured into water (2 L) and the solid which formed was collected, washed with ether (1 L) and air-dried ; yield 41.2 g (67%), m.p. 154-155° C.

d) N-[3-(Hydroxymethyl)-4-methoxyphenyl]-2-thiophenecarboximidamide

To a stirred suspension of lithium aluminium hydride (9.8 g, 260 mmol) in dry tetrahydrofuran (40 mL) at 0° C. under nitrogen was added dropwise a solution containing methyl 5-{[imino(2-thienyl)methyl]amino}-2-methoxybenzoate (38.2 g, 130 mmol) in tetrahydrofuran (100 mL) and the mixture was stirred for a further 2 h at 0° C. To the cooled solution was then added water (10 mL), followed by 15% aqueous ammonia (10 mL) and finally water (10 mL). The aluminium salts were filtered off and the solvent evaporated to afford a light yellow solid (24 g). The material was slurried in ether and the product collected; yield of nearly colourless solid (23.4 g, 68.8%); m.p. 191-192° C.

e) N-[3-(Chloromethyl)-4-methoxyphenyl]-2-thiophenecarboximidamide Hydrochloride

To a stirred solution containing N-[3-(hydroxyrnethyl)-4-methoxyphenyl]-2-thiophenecarboximidamide (14.2 g, 54 mmol) dissolved in dichloromethane (200 mL) was added dropwise thionyl chloride (19.3 g, 160 mmol) and the mixture was stirred at room temperature for 2 h. The mixture was poured into diethyl ether (1.5 L), stirred for 16 h, and the precipitate collected, washed well with diethyl ether and air-dried; yield 15.4 g (97%), m.p. 204-205° C.

### PREPARATION 8

N-[3-(Chloromethyl)-4-(1-ethylpropoxy)phenyl]-2-thiophenecarboximidamide Hydrochloride

a) 2-(1-Ethylpropoxy)-5-nitrobenzoic acid

To sodium hydride (4.5 g, 60% in oil; 106 mmol) suspended in DMSO (100 mL) was added 2-chloro-5-nitrobenzoic acid (10 g. 48 mmol) followed by 3-pentanol (6 mL, 55 mmol) and the reaction mixture was stirred at 60° C. under nitrogen for 5 days. The mixture was allowed to cool to room temperature, made acidic by the dropwise addition of 2N hydrochloric acid and extracted with ethyl acetate (3×100 mL). The organic extracts were combined, dried over magnesium sulphate and evaporated to afford an oil (15 g). The oil was chromatographed on silica gel using chloroform/methanol, 95:5, as eluent to afford the title compound (10.4 g, 85%) as an off-white solid; MS:^{m}/z 254 [M+H]⁺.

b) Methyl 2-(1-Ethylpropoxy)-5-nitrobenzoate

To a solution containing 2-(1-ethylpropoxy)-5-nitrobenzoic acid (10.4 g, 41 mmol) in anhydrous methanol (200 mL) was added thionyl chloride (3.7 mL, 51 mmol) dropwise with stirring. The resulting solution was heated at reflux for 3 h, cooled to room temperature and the solvent evaporated to afford the title compound (11 g, 100%) as a light yellow oil; MS:^{m}/z 268 [M+H]⁺.

c) Methyl 5-Amino-2-(1-ethyloropoxy)benzoate

To a solution containing methyl 2-(1-ethylpropoxy)-5-nitrobenzoate (11 g, 41 mmol) in 95% ethanol (150 mL) was added 10% Pd/C (100 mg) and the mixture was hydrogenated in a Parr apparatus at an initial pressure of 45 psi for 1 h. The catalyst was filtered off and the filtrate concentrated to give a dark oil (9.2 g). The oil was chromatographed on silica gel using ethyl acetate/hexane, 2:3, as eluent to afford the title compound (7.7 g, 80%) as a colourless oil; MS:^{m}/z 238 [M+H]⁺.

d) Methyl 2-(1-Ethylpropoxy)-5-{[imino(2-thienyl)methyl]amino}benzoate

Following the same procedure as used in the preparation of intermediate 7(c) above but substituting methyl 5-amino-2-(1-ethylpropoxy)benzoate for methyl 5-amino-2-methoxybenzoate hydrochloride the title compound was prepared in 90% yield; MS:^{m}/z 347 [M+H]⁺.

e) N-[4-(1-Ethylpropoxy)-3-(hydroxymethyl)phenyl]-2-thioiphenecarboximidamide

Following the same procedure as used in the preparation of intermediate 7(d) above but substituting methyl 2-(1-ethylpropoxy)-5-{[imino(2-thienyl)methyl]amino}benzoate for methyl 5-{imino(2-thienyl)methyl]amino}-2-methoxybenzoate the title compound was prepared in 92% yield; MS:^{m}/z 319 [M+H]⁺.

f) N-[3-(Chloromethyl)-4-(1-ethylpropoxy)phenyl]-2-thiophenecarboximidamide Hydrochloride

Following the same procedure as used in the preparation of intermediate 7(e) above but substituting N-[4-(1-ethylpropoxy)-3-(hydroxymethyl)phenyl]-2-thiophenecarboximidamide for N-[3-(hydroxymethyl)-4-methoxyphenyl]-2-thiophenecarboxiridamide the title compound was prepared in 90% yield; MS:^{m}/z 337 [M+H]⁺.

### PREPARATION 9

N-[3-(Chloromethyl)-4-isopropoxyphenyl]-2-thiophenecarboximidamide Hydrochloride

a) 2-Isopropoxy-5-nitrobenzoic acid

Following the same procedure as used in the preparation of intermediate 8(a) above but substituting 2-propanol for 3-pentanol the title compound was prepared in 70% yield; MS: ^{m}/z 226 [M+H]⁺.

b) Methyl 2-Isopropoxy-5-nitrobenzoate

Following the same procedure as used in the preparation of intermediate 8(b) above but substituting 2-isopropoxy-5-nitrobenzoic acid for 2-(1-ethylpropoxy)-5-nitrobenzoic acid the title compound was obtained as a yellow solid in 93% yield; MS:^{m}/z 240 [M+H]⁺.

c) Methyl 5-Amino-2-Isopropoxybenzoate

Following the same procedure as used in the preparation of intermediate 8(c) above but substituting methyl 2-isopropoxy-5-nitrobenzoate for methyl 2-(1-ethylpropoxy)-5-nitrobenzoate the title compound was prepared in quantitative yield; MS:^{m}/z 210 [M+H]⁺.

d) Methyl 2-Isopropoxy-5-{[imino(2-thienyl)methyl]amino}benzoate

Following the same procedure as used in the preparation of intermediate 7(c) above but substituting methyl 5-amino-2-isopropoxybenzoate for methyl 5-amino-2-methoxybenzoate hydrochloride the title compound was prepared in 93% yield; MS:^{m}/z 319 [M+H]⁺.

e) N-[3-(Hydroxymethyl)-4-isopropoxyphenyl]-2-thiophenecarboximidamide

Following the same procedure as used in the preparation of intermediate 7(d) above but substituting methyl 2-isopropoxy-5-{[imino(2-thienyl)methyl]amino}benzoate for methyl 5-}imino(2-thienyl)methyl]amino}-2-methoxybenzoate the title compound was obtained as an off-white solid in 95% yield; MS:^{m}/z 291 M+H]⁺.

f) N-[3-(Chloromethyl)-4-isopropoxyphenyl]-2-thiophenecarboximidamide Hydrochloride

Following the same procedure as used in the preparation of intermediate 7(e) above but substituting N-[3-(hydroxymethyl)-4-isopropoxyphenyl]-2-thiophenecarboximidamide for N-[3-(hydroxymethyl)-4-methoxyphenyl]-2-thiophenecarboximidamide the title compound was prepared in 90% yield; MS:^{m}/z 309 [M+H]⁺.

### PREPARATION 10

N-[3-(Chloromethyl)-4-(cyclopentyloxy)phenyl]-2-thiophenecarboximidamide hydrochloride

a) 2-Cyclopentyloxy-5-nitrobenzoic acid

Following the same procedure as used in the preparation of intermediate 8(a) above but substituting cyclopentanol for 3-pentanol the title compound was prepared in 82% yield; MS:^{m}/z 252 [M+H]⁺.

b) Methyl 2-Cyclopentyloxy-5-nitrobenzoate

Following the same procedure as used in the preparation of intermediate 8(b) above but substituting 2-cyclopentyloxy-5-nitrobenzoic acid for 2-(1-ethylpropoxy)-5-nitrobenzoic acid the title compound was prepared in 95% yield; MS:^{m}/z 266 [M+H]⁺.

c) Methyl 5-Amino-2-Cyclopentyloxybenzoate

Following the same procedure as used in the preparation of intermediate 8(c) above but substituting methyl 2-cyclopentyloxy-5-nitrobenzoate for methyl 2-(1-ethylpropoxy)-5-nitrobenzoate the title compound was prepared in quantitative yield; MS:^{m}/z 236 [M+H]⁺.

d) Methyl 2-Cyclopentyloxy-5-{[imino(2-thienyl)methyl]amino}benzoate

Following the same procedure as used in the preparation of intermediate 7(c) above but substituting methyl 5-amino-2-cyclopentyloxybenzoate for methyl 5-amino-2-methoxybenzoate hydrochloride the title compound was prepared in 88% yield; MS:^{m}/z 345 [M+H]⁺.

e) N-[4-Cyclopentyloxy-3-(hydroxymethyl)phenyl]-2-thiophenecarboximidamide

Following the same procedure as used in the preparation of intermediate 7(d) above but substituting methyl 2-cyclopentyloxy-5-{[imino(2-thienyl)methyl]amino}benzoate for methyl 5-{imino(2-thienyl)methyl]amino}-2-methoxybenzoate the title compound was prepared in 80% yield; MS:^{m}/z 317 [M+H]⁺.

f) N-[3-(Chloromethyl)-4-cyclopentyloxyphenyl]-2-thiophenecarboximidamide Hydrochloride

Following the same procedure as used in the preparation of intermediate 7(e) above but substituting N-[4-cyclopentyloxy-3-(hydroxmethyl)phenyl]-2-thiophenecarboximidamide for N-[3-(hydroxymethyl)-4-methoxyphenyl]-2-thiophenecarboximidamide the title compound was prepared in 30% yield: MS:^{m}/z 335 [M+H]⁺.

### PREPARATION 11

N-[3-(Chloromethyl)-4-phenoxyphenyl]-2-thiophenecarboximidamide Hydrochloride

a) 5-Nitro-2-phenoxybenzoic Acid

Following the same procedure as used in the preparation of intermediate 8(a) above but substituting phenol for 3-pentanol the title compound was obtained as a light brown solid in 78% yield; MS:^{m}/z 260 [M+H]⁺.

b) Methyl 5-Nitro-2-phenoxybenzoate

Following the same procedure as used in the preparation of intermediate 8(b) above but substituting 5-nitro-2-phenoxybenzoic acid for 2-(1-ethylpropoxy)-5-nitrobenzoic acid the title compound was obtained as a light tan solid in 96% yield; MS:^{m}/z 274 [M+H]⁺.

c) Methyl 5-Amino-2-phenoxybenzoate

Following the same procedure as used in the preparation of intermediate 8(c) above but substituting methyl 5-nitro-2-phenoxybenzoate for methyl 2-(1-ethylpropoxy)-5-nitrobenzoate the title compound was obtained as a colourless solid in quantitative yield; MS:^{m}/z 244 [M+H]⁺.

d) Methyl 5-{[imino(2-thienyl)methyl]amino}-2-phenoxybenzoate

Following the same procedure as used in the preparation of intermediate 7(c) above but substituting methyl 5-amino-2-phenoxybenzoate for methyl 5-amino-2-methoxybenzoate hydrochloride the title compound was obtained as a cream-coloured solid in 78% yield; MS:^{m}/z 353 [M+H]⁺.

e) N-[3-(Hydroxymethyl)-4-phenoxyphenyl]-2-thiophenecarboximidamide

Following the same procedure as used in the preparation of intermediate 7(d) above but substituting methyl 5-{[imino(2-thienyl)methyl]amino}-2-phenoxybenzoate for methyl 5-}imino(2-thienyl)methyl]amino}-2-methoxybenzoate the title compound was obtained as a light yellow solid in 80% yield; MS:^{m}/z 325 [M+H]⁺.

f) N-[3-(Chloromethyl)-4-phenoxyphenyl]-2-thiophenecarboximidamide Hydrochloride

Following the same procedure as used in the preparation of intermediate 7(e) above but substituting N-[3-(hydroxymethyl)-4-phenoxyphenyl]-2-thiophenecarboximidamide for N-[3-(hydroxymethyl)-4-methoxyphenyl]-2-thiophenecarboximidamide the title compound was obtained as a tan solid in 55% yield; MS:^{m}/z 343 [M+H]⁺.

### PREPARATION 12

N-[5-(Chloromethyl)-2-methoxyphenyl]-2-thiophenecarboximidamide Hydrochloride

a) Methyl 4-Methoxy-3-nitro-benzoate

Following the same procedure as used in the preparation of intermediate 8(b) above but substituting 4-methoxy-3-nitrobenzoic acid for 2-(1-ethylpropoxy)-5-nitrobenzoic acid the title compound was prepared in 81% yield; MS:^{m}/z 212 [M+H]⁺.

b) Methyl 3-Amino-4-methoxybenzoate

Following the same procedure as used in the preparation of intermediate 8(c) above but substituting methyl 4-methoxy-3-nitrobenzoate for methyl 2-(1-ethylpropoxy)-5-nitrobenzoate the title compound was prepared in quantitative yield; MS:^{m}/z 182 [M+H]⁺.

c) Methyl 3-{[Imino(2-thienyl)methyl]amino}-4-methoxybenzoate

Following the same procedure as used in the preparation of intermediate 7(c) above but substituting methyl 3-amino-4-methoxybenzoate for methyl 5-amino-2-methoxybenzoate hydrochloride the title compound was prepared in 33% yield; MS:^{m}/z 291 [M+H]⁺.

d) N-[5-(Hydroxymethyl)-2-methoxyphenyl]-2-thiophenecarboximidamide

Following the same procedure as used in the preparation of intermediate 7(d) above but substituting methyl 3-{[imino(2-thienyl)methyl]amino}-4-methoxybenzoate for methyl 5-{imino (2-thienyl)methyl]amino}-2-methoxybenzoate the title compound was prepared in 97% yield; MS:^{m}/z 263 [M+H]⁺.

e) N-[5-(Chloromethyl)-2-methoxyphenyl]-2-thiophenecarboximidamnide Hydrochloride

Following the same procedure as used in the preparation of intermediate 7(e) above but substituting N-[5-(hydroxymethyl)-2-methoxyphenyl]-2-thiophenecarboximidainide for N-[3-(hydroxymethyl)-4-methoxyphenyl]-2-thiophenecarboximidamide the title compound was prepared in 90% yield; MS:^{m}/z 281 [M+H]⁺.

### PREPARATION 13

5-{[Imino(2-thienyl)methyl]amino}-2-methoxy-N-methylbenzamide Hydrochloride

a) 2-Methoxy-N-methyl-5-nitrobenzamide

To sodium methoxide (1.05 g, 19.6 mmol) in methanol (50 mL) was added dropwise with cooling a solution of 2-chloro-N-methyl-5-nitrobenzamide (4.2 g, 19.6 mmol) in methanol (50 mL) and the mixture was stirred at 60° C. under nitrogen for 16 h. The methanol was removed under reduced pressure and the solid residue partitioned between chloroform/water (1:1, 100 mL). The chloroform layer was separated, washed with water (2×25 mL), and dried over magnesium sulphate. The solvent was evaporated off to afford the title compound (4.1 g, 95%) as a yellow solid; MS:^{m}/z 211 [M+H]⁺.

b) 5-Amino-2-methoxy-N-methylbenzamide Hydrochloride

Following the procedure described in Example 1(b) the above nitro compound was reduced to the free base amine in 95% yield, which was converted into the title compound on treatment of an ethanol solution with diethyl ether/hydrogen chloride; MS:^{m}/z 181 [M+H]⁺.

c) 5-{[Imino(2-thienyl)methyl]amino}-2-methoxy-N-methylbenzamide Hydrochloride

Using the method described in Example 1(c) but substituting 5-amino-2-methoxy-N-methylbenzamide hydrochloride for 4-methoxy-3-[(methylamino)-methyl]aniline hydrochloride the title compound was obtained as a colourless solid in 27% yield; MS: ^{m}/z 290 [M+H]⁻.

### PREPARATION 14

5-{[Imino(2-thienyl)methyl]amino}-2-propoxy-N-methylbenzamide Hydrochloride

a) 5-Amino-2-protoxy-N-methylbenzamide Hydrochloride

Following the procedure described in Example 1(b) the nitro compound in Example 5(a) was reduced to the free base amine in 95% yield, which was converted into the title compound on treatment of an ethanol solution with diethyl ether/hydrogen chloride; MS: ^{m}/z 209 [M+H]⁺.

b) 5-{[Imino(2-thienyl)methyl]amino}-2-propoxy-N-methylbenzamide Hydrochloride

Using the method described in Example 1(c) but substituting 5-amino-2-propoxy-N-methylbenzamide hydrochloride for 4-methoxy-3-[(methylamino)-methyl]aniline hydrochloride the title compound was obtained as a colourless solid in 50% yield; MS: ^{m}/z 318 [M+H]⁺.

### PREPARATION 15

N-(2-Fluoroethyl)-5-{[imino(2-thienyl)methyl]amino}-2-methoxybenzamide

a) 2-Chloro-N-(2-fluoroethyl)-5-nitrobenzamide

To a solution of 2-chloro-5-nitrobenzoyl chloride (11 g, 50 mmol) in dichloromethane (50 mL) cooled to 0° C. was added an ice-cold solution of dichloromethane (20 mL) containing 2-fluoroethylamine (3.1 g, 50 mmol). The reaction mixture was stirred in the cold for 1 h, diluted with methylene chloride (100 mL), and the methylene chloride solution washed sequentially with water (100 mL), 5% aqueous hydrochloric acid (100 mL), water (100 mL), aqueous saturated sodium chloride solution (2×75 mL). The methylene chloride solution was then dried over magnesium sulphate and the solvent evaporated to afford an off-white solid (10.5 g). Chromatography on silica gel using chloroform as eluent afforded the title compound (6.5 g, 52.4%) as a colourless solid; MS: ^{m}/z 247 [M+H]⁺.

b) N-(2-Fluoroethyl)-2-methoxy-5-nitrobenzamide

To sodium methoxide (2.5 g, 46.8 mmol) in methanol (100 mL) was added dropwise with cooling a solution of 2-chloro-N-(2-fluoroethyl)-5-nitrobenzamide (6.5 g, 26.4 mmol) in methanol (50 mL) and the mixture was stirred at 60° C. under nitrogen for 16 h. The methanol was removed under reduced pressure and the solid residue partitioned between chloroform/water (1:1, 100 mL). The chloroform layer was separated, washed with water (2×25 mL), and dried over magnesium sulphate. The solvent was evaporated off to afford the title compound (4.9 g, 77%) as a light yellow solid; MS:^{m}/z 243 [M+H]⁺.

c) 5-Amino-N-(2-fluoroethyl)-2-methoxybenzamide

Following the procedure described in Example 1(b) the above nitro compound was reduced to the title compound in 95% yield; MS:^{m}/z 213 [M+H]⁻.

d) N-(2-Fluoroethyl)-5-{[imino(2-thienyl)methyl]amino}-2-methoxybenzamide

Using the method described in Example 1(c) but substituting 5-amino-N-(2-fluoroethyl)-2-methoxybenzamide for 4-methoxy-3-[(methylamino)-methyl]aniline hydrochloride the title compound was obtained as a colourless glassy solid in 40% yield; MS:^{m}/z 322 [M+H]⁺.

### PREPARATION 16

N-(3-Acetyl-4-methoxyphenyl)-2-thiophenecarboximidamide

(a) N-(3-Acetyl-4-methoxylphenyl)butanamide

N-(3-Acetyl-4-hydroxyphenyl)butanamide (20.9 g, 95 mmol) and potassium carbonate (42.5 g, 308 mmol) were stirred in DMF (200 ml) under nitrogen. Iodomethane (12 ml, 27.3 g, 192 mmnol) was added, and stirring was continued overnight. The solution was evaporated, and the residue was partitioned between ethyl acetate and water. The organic layer was dried (magnesium sulfate), filtered, and recrystallised from ethanol to give the sub-title compound (16.9 g, 72 mmol, 76%) as a colourless solid. m.p. 113° C.; MS (ES⁺) ^{m}/z 236 (100%, MH⁺).

(b) 1-(5-Amino-2-methoxyphenyl)ethanone

N-(3-Acetyl-4-methoxyphenyl)butanamide (3.00 g, 12.8 mmol) was dissolved in a 1:1 mixture of concentrated hydrochloric acid and water. The solution was stirred at 100° C. for 1 h. The solution was allowed to cool, then basified with aqueous sodium hydroxide and extracted with dichloromethane. The organic layer was dried (magnesium sulfate), filtered, and evaporated to give the sub-title compound as an oil (1.96 g, 11.9 mmol, 93%). A sample of the compound was dissolved in methanol, excess hydrogen chloride (4M in dioxane) was added, then the solution was evaporated giving the hydrochloride salt of the sub-title compound as a solid. MS (ES⁺) ^{m}/z 166 (MH⁺).

(c) N-(3-Acetyl-4-methoxyphenyl)-2-thiophenecarboximidamide

A solution containing 1-(5-amino-2-methoxyphenyl)ethanone (1.54 g, 9.3 mmol) and 2-thiophenecarboximidothioic acid ethyl ester hydrochloride (2.24 g, 10.8 mmol) in ethanol (30 ml) was heated at 60° C. under nitrogen overnight. The solution was evaporated. The residue was stirred with aqueous potassium carbonate for about 1 h. The resulting solid was collected by filtration, washed with water, then dried under vacuum. Recrystallisation from ethyl acetate/hexane gave the title compound as a pale solid (1.92 g, 7.00 mmol, 75%). MS (ES⁻) ^{m}/z 275 (100%, MH⁺).

### PREPARATION 17

1-(5-Bromo-2-methoxyphenyl)-4-chloro-1-butanone

A mixture of 4-chlorobutanoyl chloride (25 ml, 31.45 g, 250 mmol) and 4-bromoanisole (25 ml, 37.35 g, 200 mmol) was added dropwise to a solution of aluminium chloride in nitrobenzene (1M, 250 ml, 250 mmol) which was stirred at 0° C. under nitrogen. Stirring was continued overnight and the solution was allowed to warm slowly to room temperature. The solution was poured into ice, and the resulting solution was then evaporated under vacuum. The residue was dissolved in ether, and the solution was filtered. Rotary evaporation of the ether followed by Kugelrohr distillation of the residue (280° C., oven temperature) gave the title compound as an oil. MS (ES⁺) ^{m}/z 290, 292, 294 (MH⁺).

### EXAMPLE 1

N-{4-Methoxy-3-[(methylamino)methyl]phenyl}-2-thiophenecarboximidamide Dihydrochloride

a) (2-Methoxy-5-nitrophenyl)-N-methylmethanamine Hydrochloride

To 2-methoxy-5-nitrobenzaldehyde (4.9 g, 27 mmol) dissolved in methanol (200 mL) was added a solution of methylamine (1.26 g, 40 mmol) dissolved in methanol (20 mL) containing acetic acid (2.4 g, 40 mmol), followed immediately by sodium cyanoborohydride (2.55 g, 40 mmol). The reaction mixture was stirred at room temperature under nitrogen for 16 h, the methanol evaporated off, and the residue taken up in ethyl acetate (250 mL). The ethyl acetate solution was treated dropwise with 1M aqueous potassium hydrogen sulphate until pH 3 to 4, then with saturated aqueous sodium bicarbonate solution until pH 7 and the layers were then separated. The aqueous phase was extracted with additional ethyl acetate (4×50 mL). The ethyl acetate solutions were combined, washed with saturated aqueous sodium chloride solution, dried (magnesium sulphate) and evaporated to afford a solid. Chromatography on silica gel using a gradient of 10-40% ethyl acetate in methanol afforded the free base (611 mg, 11.5%) of the title compound as a yellow solid. Dissolution in ethanol and treatment with a saturated ethanolic solution of hydrogen chloride afforded the title compound as a pale yellow solid; MS:^{m}/z 197 [M+H]⁺.

b) 4-Methoxy-3-[(methylamino)methyl]aniline Hydrochloride

To N-methyl-(2-methoxy-5-nitrophenyl)methanamine hydrochloride (600 mg) in 95% ethanol (60 mL) was added a catalytic amount (˜30 mg) of 10% Pd/C and the mixture was hydrogenated at 45 psi until the required amount of hydrogen was taken up (approximately 2 h). The catalyst was filtered off and the solvent evaporated to afford the title compound (510 mg, 98%); MS:^{m}/z 167 [M+H]⁺.

c) N-{4-Methoxy-3-[(methylamino)methyl]phenyl}-2-thiophenecarboximidamide Dihydrochloride

To 4-methoxy-3-[(methylamino)methyl]aniline hydrochloride (920 mg, 4.5 mmol) in ethanol (5 mL) was added 2-thiophenecarboximidothioic acid ethyl ester hydrochloride (1.6 g, 5.6 mmol) and the solution was stirred under nitrogen with heating to 60° C. for 6 h. Water (15 mL) was then added, the solution basified to pH 11 with aqueous ammonia, and the aqueous solution extracted with ethyl acetate (3×100 mL). The ethyl acetate extracts were combined, dried over magnesium sulphate and evaporated to afford a viscous oil (1.4 g). Chromatography on silica gel using chloroform/methanol, 8:2, afforded the free base of the title compound as a light tan solid. Dissolution in 2-propanol and acidification with diethyl ether/hydrogen chloride afforded the title compound (820 mg, 51.6%) as an off-white solid; MS:^{m}/z 276 [M+H]⁺.

### EXAMPLE 2

N-[4-Methoxy-3-(1-pyrrolidinylmethyl)phenyl}-2-thiophenecarboximidamide Dihydrochloride

a) 1-(2-Methoxy-5-nitrobenzyl)pyrrolidine Hydrochloride

Using the method described in Example 1(a) but substituting pyrrolidine for methylamine the title compound was obtained as a yellow solid in 35% yield; MS:^{m}/z 237 [M+H]⁻.

b) 4-Methoxy-3-(1-pyrrolidinylmethyl)aniline Hydrochloride

Following the procedure described in Example 1(b) the nitro compound obtained in Example 2(a) was reduced to the title compound in 95% yield; MS:^{m}/z 207 [M+H]⁺.

c) N-[4-Methoxy-3-(1-pyrrolidinylmethyl)phenyl}-2-thiophenecarboximidamide Dihydrochloride

Using the method described in Example 1(c) but substituting 4-methoxy-3-(1-pyrrolidinylmethyl)aniline hydrochloride for 4-methoxy-3-[(methylamino)-methyl]aniline hydrochloride, the title compound was obtained as a colourless solid in 30% yield; MS:^{m}/z 316 [M+H]⁺.

### EXAMPLE 3

N-[4-Methoxy-3-(1-morpholinylmethyl)phenyl}-2-thiophenecarboximidamide Dihydrochloride

a) 1-(2-Methoxy-5-nitrobenzyl)morpholine Hydrochloride

Using the method described in Example 1(a) but substituting morpholine for methylamine, the title compound was obtained as a yellow solid in 63% yield; MS:^{m}/z 253 [M+H]⁺.

b) 4-Methoxy-3-(1-morpholinylmethyl)aniline Hydrochloride

Following the procedure described in Example 1(b) the nitro compound obtained in Example 3(a) was reduced to the title compound in 85% yield; MS:^{m}/z 223 [M+H]⁺.

c) N-[4-Methoxy-3-(1-morpholinylmethyl)phenyl}-2-thiophenecarboximidamide Dihydrochloride

Using the method described in Example 1(c) but substituting 4-methoxy-3-(1-morpholinylmethyl)aniline hydrochloride for 4-methoxy-3-[(methylamino)methyl]aniline hydrochloride, the title compound was obtained as a colourless solid in 52% yield; MS:^{m}/z 332 [M+H⁺.

### EXAMPLE 4

N-{4-Methoxy-3-[(4-methyl-1-piperazinyl)methyl]phenyl}-2-thiophenecarboximidamide Dihydrochloride

a) 1-(2-Methoxy-5-nitrobenzyl)-4-methylpiperazine

To 2-methoxy-5-nitrobenzaldehyde (1.3 g, 7.2 mmol) dissolved in methanol (20 mL) was added N-methylpiperazine (2.5 g, 29 mmol) followed by sodium cyanoborohydride (0.45 g, 7.2 mmol) and zinc chloride (0.4 g, 3.5 mmol). The reaction mixture was allowed to stir at room temperature under nitrogen for 16 h and then treated with 0.1N aqueous sodium hydroxide (45 mL) with stirring. The methanol was evaporated off and the resulting aqueous solution extracted with ethyl acetate (3×75 mL). The ethyl acetate extracts were combined, dried (magnesium sulphate) and evaporated to afford the crude product (1.6 g). Chromatography on silica gel using chloroform/methanol, 95:5, afforded the title compound (700 mg, 39%) as an oil; MS:^{m}/z 266 [M+H]⁺.

b) 4-Methoxy-3-[(4-methyl-1-piperazinyl)methyl]aniline Hydrochloride

Following the procedure described in Example 1(b) the nitro compound obtained in Example 4(a) was reduced to the title compound in 90% yield; MS:^{m}/z 236 [M+H]⁺.

c) N-{4-Methoxy-3-[(4-methyl-1-piperazinyl)methyl]phenyl}-2-thiophenecarboximidamide Dihydrochloride

Using the method described in Example 1(c) but substituting 4-methoxy-3-[(4-methyl-1-piperazinyl)methyl]aniline hydrochloride for 4-methoxy-3-[(methylamino)methyl]aniline hydrochloride, the title compound was obtained as a colourless solid in 40% yield; MS:^{m}/z 345 [M+H]⁺.

### EXAMPLE 5

N-{4-Propoxy-3-[(methylamino)methyl]phenyl}-2-thiophenecarboximidamide Dihydrochloride

a) N-Methyl-5-nitro-2-propoxybenzamide

To a solution of sodium propoxide in propanol, prepared by dissolving sodium metal (0.54 g, 24 mmol) in 1-propanol (25 ml), was added, in rapid drops, a solution of N-methyl-2-chloro-5-nitrobenzamide (5 g, 24 mmol) in 2-propanol (125 mL) and the reaction mixture was heated at 60° C. for 48 h. The mixture was cooled, the solvent evaporated and the yellow solid residue dissolved in chloroform (150 mL). The chloroform solution was washed with water (2×50 mL), dried over magnesium sulphate, filtered and evaporated to afford the title compound (5.1 g, 90%) as a yellow solid; MS:^{m}/z 239 [M+H]⁺.

b) N-Methyl-(2-propoxy-5-nitrophenyl)methanamine

To an ice-cold solution of N-methyl-5-nitro-2-propoxybenzamide (0.75 g, 3.0 mmol) in tetrahydrofuran (20 mL) was added dropwise a 1 M solution of borane in tetrahydrofuran (30 mL). After all of the borane solution had been added the reaction mixture was heated at reflux under nitrogen for 8 h. The reaction mixture was then cooled, treated dropwise in the cold with 10% hydrochloric acid (10 mL), diluted with water (20 mL), and basified with aqueous sodium carbonate solution until pH 11. The solution was extracted with ethyl acetate (3×50 mL), the ethyl acetate extracts combined, dried (magnesium sulphate) and evaporated to afford an oil (0.95 g). Chromatography on silica gel using chloroform: methanol, 9:1, provided the title compound (0.45 g, 66%) as an oil; MS:^{m}/z 225 [M+H]⁺.

c) 3-[(Methylamino)methyl]-4-propoxyaniline Hydrochloride

Following the procedure described in Example 1(b), the nitro compound obtained in Example 5(b) was reduced to the free base amine in 95% yield. This material was then converted into the title compound by treatment of an ethanol solution with diethyl ether/hydrogen chloride; MS:^{m}/z 195 [M+H]⁺.

d) N-{4-Propoxy-3-[(methylamino)methyl]phenyl}-2-thiophenecarboximidamide Dihydrochloride

Using the method described in Example 1(c) but substituting 3-[(methylamino)methyl]-4-propoxyaniline for 4-methoxy-3-[(methylamino)methyl]aniline hydrochloride, the title compound was obtained as a colourless solid in 30% yield; MS:^{m}/z 304 [M+H]⁺.

### EXAMPLE 6

N-{4-(Cyclopentyloxy)-3-[(methylamino)methyl]phenyl}-2-thiophenecarboximidamide Dihydrochloride

a) 2-(Cyclolpentyloxy)-N-methyl-5-nitrobenzamide

Sodium (0.56 g, 25 mmol) and cyclopentanol (2.2 g, 26 mmol) were refluxed in toluene (50 mL) until all of the sodium dissolved. The toluene was evaporated off and the residue taken up in dimethylsulphoxide (25 mL). The resulting sodium cyclopentyloxide solution was added dropwise to a solution of 2-chloro-N-methyl-5-nitrobenzamide (5 g, 23 mmol) in dimethylsulphoxide (50 mL) and the mixture was stirred at room temperature under nitrogen for 16 h. The reaction mixture was poured into water (200 mL) and the aqueous dimethylsulphoxide solution extracted with ethyl acetate (3×100 mL). The ethyl acetate extracts were combined, washed with water (2×100 mL), aqueous sodium chloride solution (1×100 mL), dried with magnesium sulphate and filtered. The solvent was evaporated off to afford the title compound (6.0 g, 94%) as a tan solid; MS:^{m}/z 265 [M+H]⁺.

b) [2-(Cyclopentyloxy)-5-nitrophenyl]-N-methylmethanamine

Using the method described in Example 5(b) but substituting 2-(cyclopentyloxy)-N-methyl-5-nitrobenzamide for N-methyl-5-nitro-2-propoxybenzamide, the title compound was obtained as a light yellow oil in 64% yield; MS:^{m}/z 251 [M+H]⁺.

c) 4-(Cyclopentyloxy)-3-[(methylamino)methyl]aniline Hydrochloride

Following the procedure described in Example 1(b) the nitro compound obtained in Example 6(b) was reduced to the free base amine in 95% yield. This material was converted into the title compound on treatment of an ethanol solution with diethyl ether/hydrogen chloride; MS: ^{m}/z 221 [M+H]⁺.

d) N-{4-(Cyclopentyloxy)-3-[(methylamino)methyl]phenyl}-2-thiophenecarboximidamide Dihydrochloride

Using the method described in Example 1(c) but substituting 4-(cyclopentyloxy)-3-[(methylamino)methyl]aniline hydrochloride for 4-methoxy-3-[(methylamino)-methyl]aniline hydrochloride, the title compound was obtained as a colourless solid in 42% yield; MS: ^{m}/z 330 [M+H]⁺.

### EXAMPLE 7

N-[4-Cyclopentyloxy-3-(1-pyrrolidinylmethyl)phenyl}-2-thiophenecarboximidamide

MS: ^{m}/z 370 [M+H]⁺.

### EXAMPLE 8

N-[4-Methoxy-3-(1-morpholinylmethyl)phenyl}-3-thiophenecarboximidamide Dihydrochloride

MS: ^{m}/z 332 [M+H]⁺.

### EXAMPLE 9

N-{2-Methoxy-5-[(methylamino)methyl]phenyl}-2-thiophenecarboximidamide Dihydrochloride

a) (4-Methoxy-3-nitrophenyl)-N-methylmethanamine Hydrochloride

Using the method described in Example 1(a) but substituting 4-methoxy-3-nitrobenzaldehyde for 2-methoxy-5-nitrobenzaldehyde, the title compound was obtained as a yellow solid in 35% yield; MS: ^{m}/z 197 [M+H]⁺.

b) 2-Methoxy-5-[(methylamino)methyl]aniline Hydrochloride

Following the procedure described in Example 1(b) the nitro compound obtained in Example 9(a) was reduced to the title compound in 95% yield; MS: ^{m}/z 167 [M+H]⁺.

d) N-{4-Methoxy-3-[(methylamino)methyl]phenyl}-2-thiophenecarboximidamide Dihydrochloride

Using the method described in Example 1(c) but substituting 2-methoxy-5-[(methylamino)methyl]aniline hydrochloride for 4-methoxy-3-[(methylamino)-methyl]aniline hydrochloride, the title compound was obtained as a colourless solid in 49% yield; MS: ^{m}/z 276 [M+H]⁺.

### EXAMPLE 10

N-{2-Methoxy-3-[(methylamino)methyl]phenyl}-2-thiophenecarboximidamide Dihydrochloride

a) (2-Methoxy-3-nitrophenyl)-N-methylmethanamine Hydrochloride

Using the method described in Example 1(a) but substituting 2-methoxy-3-nitrobenzaldehyde for 2-methoxy-5-nitrobenzaldehyde, the title compound was obtained as a yellow solid in 22% yield; MS: ^{m}/z 197 [M+H]⁺.

b) 2-Methoxy-3-[(methylamino)methyl]aniline Hydrochloride

Following the procedure described in Example 1(b) the nitro compound obtained in Example 10(a) was reduced to the title compound in 95% yield. MS: ^{m}/z 167 [M+H]⁺.

c) N-{2-Methoxy-3-[(methylamino)methyl]phenyl}-2-thiophenecarboximidamide

Using the method described in Example 1(c) but substituting 2-methoxy-3-[(methylamino)methyl]aniline hydrochloride for 4-methoxy-3-[(methylamino)-methyl]aniline hydrochloride, the title compound was obtained as a colourless glassy solid in 42% yield; MS: ^{m}/z 276[M-H]⁺.

### EXAMPLE 11

N-{4-Methoxy-3-[(methylamino)methyl]phenyl}-2-furancarboximidamide

Using the method described in Example 1(c) but substituting 2-furancarboximidothioic acid ethyl ester hydrochloride for 2-thiophenecarboximidothioic acid ethyl ester hydrochloride, the title compound was obtained as a colourless solid in 67% yield; MS: ^{m}/z 260[M+H]⁺.

### EXAMPLE 12

N-3-{[(2-Fluoroethyl)amino]methyl}-4-methoxyphenyl)-2-thiophenecarboximidamide Hydrochloride

a) 2-Fluoro-N-(2-methoxy-5-nitrobenzyl)ethanamine

Using the method described in Example 5(b) but substituting N-(2-fluoroethyl)-2-methoxy-5-nitrobenzamide for N-methyl-5-nitro-2-propoxybenzamide, the title compound was obtained as a tan powder in 34% yield; MS: ^{m}/z 229 [M+H]⁺.

b) 3-{[(2-Fluoroethyl)amino]methyl}-4-methoxyaniline Hydrochloride

Following the procedure described in Example 1(b) the nitro compound obtained in Example 13(a) was reduced to the free base amine in 95% yield. This material was then converted into the title compound on treatment of an ethanol solution with diethyl ether/hydrogen chloride; MS: ^{m}/z 199 [M+H]⁺.

c) N-(3-{[(2-Fluoroethyl)amino]methyl}-4-methoxyphenyl)-2-thiophenecarboximidamide Hydrochloride

Using the method described in Example 1(c) but substituting 3-{[(2-fluoroethyl)amino]-methyl}-4-methoxyaniline hydrochloride for 4-methoxy-3-[(methylamino)methyl]aniline hydrochloride, the title compound was obtained as a colourless solid in 25% yield; MS: ^{m}/z 308 [M+H]⁺.

### EXAMPLE 13

N-[3-(Aminomethyl)-4-methoxyphenyl)-2-thiophenecarboximidamide

N-[3-(Chloromethyl)-4-methoxyphenyl)-2-thiophenecarboximidamide hydrochloride (629 mg, 1.98 mmol) was dissolved in 7N methanolic ammonia (20 ml). The solution was stirred overnight. The solution was evaporated and the residue was subjected to reverse phase HPLC on a Waters Bondapak® C₁₈ column using a gradient of acetonitrile and 0.1% aqueous trifluoroacetic acid as the eluent. Potassium carbonate was added to the product-containing fractions, and the mixture was then extracted with chloroform. The organic extracts were dried (magnesium sulfate), filtered, and evaporated to give the title compound as a solid (94 mg, 18%). MS: ^{m}/z 262 [M+H]⁺.

### EXAMPLE 14

N-(3-{[(2,2-Difluoroethyl)amino)methyl-4-methoxyphenyl)-2-thiophenecarboximidamide Dihydrochloride

N-[3-(Chloromethyl)-4-methoxyphenyl)-2-thiophenecarboximidamide hydrochloride (539.4 mg), 2,2-difluoroethylamine (280 mg) and diisopropylethylamine (1.6 ml) in DMF (10 ml) were stirred together at room temperature for 3 days and then heated at 60° C. for 2 h. The mixture was diluted with water. Excess potassium carbonate was added. The aqueous layer was extracted with dichloromethane then dried (magnesium sulfate), filtered and evaporated. The residue was purified by reverse phase HPLC on a Waters Bondapak® C₁₈ column using a gradient of acetonitrile and 0.1% aqueous trifluoroacetic acid as the eluent. The free base was prepared by basification of the product-containing fractions with potassium carbonate and extraction with dichloromethane. The organic extract was dried (magnesium sulfate), filtered, and evaporated. Dissolution of the residue from evaporation of the extracts in methanol, addition of excess hydrogen chloride solution (1M in diethyl ether) and evaporation gave the title compound as a colourless solid (258.1 mg); MS: ^{m}/z 326 [M+H]⁺.

### EXAMPLE 15

N-(4-Methoxy-3-{[(2,2,2-trifluoroethyl)amino]methyl}phenyl-2-thiophenecarboximidamide

Using the method described in Example 14 but substituting 2,2,2-trifluoroethylamine for 2,2-difluoroethylamine the title compound (as free base) was obtained as a colourless solid in 25% yield; MS: ^{m}/z 344 [M+H]⁺.

### EXAMPLE 16

N-(3-[(Cyclopropylamino}methyl]-4-methoxyphenyl}-2-thiophenecarboximidamide

Using the method described in Example 14 but substituting cyclopropylamine for 2,2-difluoroethylamine the title compound (as free base) was obtained as a colourless solid; MS: ^{m}/z 302 [M+H]⁺.

### EXAMPLE 17

N-{3-[(Diethylamino)methyl]-4-methoxyphenyl}-2-thiophenecarboximidamide

Using the method described in Example 14 but substituting diethylamine for 2,2-difluoroethylamine the title compound (as free base) was obtained as a colourless solid in 55% yield; MS: ^{m}/z 318 M+H]⁺.

### EXAMPLE 18

N-{3-[(Isopropylamino)methyl]-4-methoxyphenyl}-2-thiophenecarboximidamide

Using the method described in Example 14 but substituting isopropylamine for 2,2-difluoroethylamine the title compound (as free base) was obtained as a colourless solid in 27% yield; MS: ^{m}/z 304 [M+H]⁺.

### EXAMPLE 19

N-{4-Isopropoxy-3-[{methylamino}methyl]phenyl}-2-thiophenecarboximidamide

Using the method described in Example 14 but substituting methylamine for 2,2-difluoroethylamine and substituting N-[3-(chloromethyl)-4-isopropoxyphenyl]-2-thiophenecarboximidamide hydrochloride for N-[3-(chloromethyl)-4-methoxyphenyl]-2-thiophenecarboximidamide hydrochloride the title compound (as free base) was obtained as a colourless solid; MS: ^{m}/z 304 [M+H]⁺.

### EXAMPLE 20

N-{4-Isopropoxy-3-[{isopropylamino}methyl]phenyl}-2-thiophenecarboximidamide Dihydrochloride

Using the method described in Example 14 but substituting isopropylamine for 2,2-difluoroethylamine and substituting N-[3-(chloromethyl-4-isopropoxyphenyl]-2-thiophenecarboximidamide hydrochloride for N-[3-(chloromethyl)-4-methoxyphenyl]-2thiophenecarboximidamide hydrochloride the title compound was obtained as a colourless solid in 45% yield; MS: ^{m}/z 332 [M+H]⁺.

### EXAMPLE 21

N-[4-Isopropoxy-3-(1-pyrrolidinylmethyl)phenyl]-2-thiophenecarboximidamide

Using the method described in Example 14 but substituting pyrrolidine for 2,2-difluoroethylamine and substituting N-[3-(chloromethyl)-4-isopropoxyphenyl]-2-thiophenecarboximidamide hydrochloride for N-[3-(chloromethyl)-4-methoxyphenyl]-2-thiophenecarboximidamide hydrochloride the title compound (as free base) was obtained as a colourless solid; m.p. 138-140° C.; MS: ^{m}/z 344 [M+H]⁺.

### EXAMPLE 22

N-{4-(1-Ethylpropoxy)-3-[(methylamino)methyl]phenyl}-2thiophenecarboximidamide

Using the method described in Example 14 but substituting methylamine for 2,2-difluoroethylamine and substituting N-[3-(chloromethyl)4-(1-ethylpropoxy)phenyl]-2-thiophenecarboximidamide hydrochloride for N-[3-(chloromethyl)-4-methoxyphenyl]-2-thiophenecarboximidamide hydrochloride the title compound (as free base) was obtained as a colourless solid in 20% yield; MS: ^{m}/z 332 [M+H]⁻.

### EXAMPLE 23

N-{4-(1-Ethylpropoxy)-3-[(isopropylamino)methyl]phenyl}-2thiophenecarboximidamide Dihydrochloride

Using the method described in Example 14 but substituting isopropylamine for 2,2-difluoroethylamine and substituting N-[3-(chloromethyl)-4-(1-ethylpropoxy)phenyl]-2-thiophenecarboximidamide hydrochloride for N-[3-(chloromethyl)-4-methoxyphenyl]-2-thiophenecarboximidamide hydrochloride the title compound was obtained as a yellow solid in 85% yield; MS: ^{m}/z 360 [M+H]⁺.

### EXAMPLE 24

N-{4-Cyclopentyloxy-3-[(isopropylamino)methyl]phenyl}-2-thiophenecarboximidamide Dihydrochloride

Using the method described in Example 14 but substituting isopropylamine for 2,2-difluoroethylamine and substituting N-[3-(chloromethyl)-4-cyclopentyloxyphenyl]-2-thiophenecarboximidamide hydrochloride for N-[3-(chloromethyl)-4-methoxyphenyl]-2-thiophenecarboximidamide hydrochloride the title compound was obtained as a yellow solid in 25% yield; MS: ^{m}/z 358 [M+H]⁺.

### EXAMPLE 25

N-{3-[(Methylamino)methyl]-4-phenoxyphenyl}-2-thiophenecarboximidamide Dihydrochloride

Using the method described in Example 14 but substituting methylamine for 2′,2-difluoroethylamine and substituting N-[3-(chloromethyl)-4-phenoxyphenyl]-2-thiophenecarboximidamide hydrochloride for N-[3-(chloromethyl)-4-methoxyphenyl-2-thiophenecarboximidamide hydrochloride the title compound was obtained as a colourless solid in 33% yield; MS: ^{m}/z 338 [M+H]⁺.

### EXAMPLE 26

N-{3-[(Isopropylamino)methyl]-4-phenoxyphenyl}-2-thiophenecarboximidamide Dihydrochloride

Using the method described in Example 14 but substituting isopropylamine for 2,2-difluoroethylamine and substituting N-[3-(chloromethyl)-4-phenoxyphenyl]-2-thiophenecarboximidamide hydrochloride for N-[3-(chloromethyl)-4-methoxyphenyl]-2-thiophenecarboximidamide hydrochloride the title compound was obtained as a colourless solid in 32% yield; MS: ^{m}/z 366 [M+H]⁺.

### EXAMPLE 27

N-{3-[(Cyclopropylamino)methyl]-4phenoxyphenyl}-2-thiophenecarboximidamide Dihydrochloride

Using the method described in Example 14 but substituting cyclopropylamine for 2,2-difluoroethylamine and substituting N-[3-(chloromethyl)-4-phenoxyphenyl]-2-thiophenecarboximidamide hydrochloride for N-[3-(chloromethyl)-4-methoxyphenyl]-2-thiophenecarboximidamide hydrochloride the title compound was obtained as a colourless solid in 30% yield; MS: ^{m}/z 364 [M+H]⁺.

### EXAMPLE 28

N′-(3-{[(2-fluoroethyl)amino]methyl}-4-phenoxyphenyl)-2-thiophenecarboximidamide Dihydrochloride

Using the method described in Example 14 but substituting 2-fluoroethylamine for 2,2-difluoroethylamine and substituting N-[3-(chloromethyl)-4-phenoxyphenyl]-2-thiophenecarboximidamide hydrochloride for N-[3-(chloromethyl)-4-methoxyphenyl]-2-thiophenecarboximidamide hydrochloride the title compound was obtained as a colourless solid in 32% yield; MS : ^{m}/z 370 [M+H]⁺.

### EXAMPLE 29

N-{4-Methoxy-3-[1-(methylamino)ethyl]phenyl}-2thiophenecarboximidamide

N-(3-Acetyl-4-methoxyphenyl)-2-thiophenecarboximidamide (283 mg, 1.03 mmol) was dissolved in a solution prepared by dissolving acetic acid (1.25 ml, 1.31 g, 21.8 mmol) in methanolic methylamine (2M, 10 ml, 20 mmol). After about 1 h, sodium cyanoborohydride (96 mg, 1.53 mmol) was added and stirring was continued overnight. The solution was evaporated. In a fume hood, hydrochloric acid was added to the residue. After about 1 h, the solution was basified with aqueous sodium hydroxide. A solid precipitated which was collected by filtration and recrystallised from t-butyl methyl ether/hexane to give the title compound as a pale solid (49 mg, 0.17 mmol, 16%). MS (ES⁻) ^{m}/z 290 (100%, MH⁻).

### EXAMPLE 30

N-[4-Methoxy-3-(1-methyl-2-pyrrolidinyl)phenyl]-2-thiophenecarboximidamide

(a) 4-Bromo-2-(1-methyl-2-pyrrolidinyl)phenyl Methyl Ether

1-(5-Bromo-2-methoxyphenyl)-4-chloro-1-butanone (5.02 g, 17.2 mmol) was dissolved in a solution prepared by adding acetic acid (5.3 ml, 5.55 g 93 mmol) to a solution of methanolic methylamine (2M, 43 ml, 86 mmol). After about 4 h, sodium cyanoborohydride (1.70 g, 27.0 mmol) was added and stirring was continued overnight. The solution was evaporated. In a fume hood, hydrochloric acid was added to the residue. After about 1 h, the solution was basified with aqueous sodium hydroxide, and then extracted with dichloromethane. The organic extract was dried (magnesium sulfate), filtered, and evaporated. The product was purified by reverse phase HPLC on a Waters Bondapak® C₁₈ column using a gradient of acetonitrile and 0.1% aqueous trifluoroacetic acid as the eluent. The product-containing fractions were evaporated. The residue was dissolved in aqueous hydrochloric acid, and the solution was evaporated. The residue was co-evaporated with ethanol and then with t-butyl methyl ether to give the hydrochloride salt of the sub-title compound as a solid (1.74 g, 5.67 mmol, 33%). MS (ES⁻) ^{m}/z 270, 272 (MH⁺).

(b) N-[4-Methoxy-3-(1-methyl-2-pyrrolidinyl)phenyl]-2-thiophenecarboximidamide

The hydrochloride salt of 4-bromo-2-(1-methyl-2-pyrrolidinyl)phenyl methyl ether (1.74 g, 5.67 mmol) was converted into the free base by partitioning between aqueous sodium carbonate and chloroform. The organic layer was dried with magnesium sulfate and passed through a short silica gel column, washing through with ethyl acetate. The solution was then evaporated and dried under vacuum. In a dry tube, a solution of the 4-bromo-2-(1-methyl-2-pyrrolidinyl)phenyl methyl ether free base, tris(dibenzylidineacetone)dipalladium (0) (77 mg, 0.084 mmol), racemic 2,2′-bis(diphenylphosphino)-1,1′-binaphthyl (125 mg, 0.20 mmol), sodium t-butoxide (2.12 g, 22.1 mmol), and benzophenone imine (1.90 ml. 2.05 g, 11.3 mmol) in tetrahydrofuran (40 ml) was sealed under nitrogen. The solution was then stirred at 100° C. (bath temperature) overnight. The solution was then allowed to cool, and was evaporated. The residue was dissolved in tetrahydrofuran (200 ml) and 6N hydrochloric acid (50 ml) was added. After 2 h, the solution was evaporated, the residue was partitioned between aqueous sodium carbonate and dichloromethane, and the organic extract was dried (magnesium sulfate), filtered, and evaporated. To the residue, 2-thiophenecarboximidothioic acid ethyl ester hydrochloride (4.66 g, 29.4 mmol), ethanol (50 ml), and diisopropylethylamine (5 ml) were added, and the solution was heated under nitrogen at 80° C. (bath temperature) overnight. The solution was evaporated, the residue was partitioned between sodium carbonate and dichloromethane, and the organic extract was dried (magnesium sulfate), filtered, and evaporated. The product was purified by reverse phase HPLC on a Waters Bondapak® C₁₈ column using a gradient of acetonitrile and 0.1% aqueous trifluoroacetic acid as the eluent. The product-containing fractions were evaporated. The residue was dissolved in aqueous hydrochloric acid, and the solution was evaporated. The residue was co-evaporated with ethanol and then with t-butyl methyl ether to give the dihydrochloride salt of the title compound as a solid (242 mg, 0.62 mmol. 11%). MS (ES⁺) ^{m}/z 316 (100%, MH⁻).

### EXAMPLE 31

N-[4-Methoxy-3-(2-pyrrolidinyl)phenyl]-2-thiophenecarboximidamide

(a) 4-Bromo-2-(2-pyrrolidinyl)phenyl Methyl Ether

1-(5-Bromo-2-methoxyphenyl)-4-chloro-1-butanone (3.04 g, 10.4 mmol) was dissolved in a solution of ammonium acetate (3.90 g, 51 mmol) in methanol (20 ml). After about 16 h. sodium cyanoborohydride (942 mg, 15.0 mmol) was added, and stirring was continued overnight. The solution was evaporated. In a fume hood, hydrochloric acid was added to the residue. After about 1 h, the solution was basified with aqueous sodium hydroxide, and then extracted with chloroform. The organic extract was dried (magnesium sulfate), filtered, and evaporated. The product was purified by reverse phase HPLC on a Waters Bondapak® C₁₈ column using a gradient of acetonitrile and 0.1% aqueous trifluoroacetic acid as the eluent. The product-containing fractions were evaporated. The residue was dissolved in aqueous hydrochloric acid, and the solution was evaporated. The residue was co-evaporated with ethanol and then with t-butyl methyl ether to give the hydrochloride salt of the sub-title compound as a solid (494 mg, 1.69 mmol, 16%). MS (ES⁻) ^{m}/z 256, 258 (MH⁺).

(b) N-[4-methoxy-3-(2-pyrrolidinyl)phenyl]-2-thiophenecarboximidamide

Di-t-butyl dicarbonate (0.80 ml, 0.76 g, 3.5 mmol) was added to a solution of 4-bromo-2-(2-pyrrolidinyl)phenyl methyl ether hydrochloride (246 mg, 0.842 mmol) and triethylamine (0.24 ml, 0.17 g, 1.72 mmol) in dry methanol (5 ml). The solution was stirred overnight at room temperature. The solution was then evaporated and the residue was partitioned between aqueous sodium carbonate and chloroform. The organic layer was dried with magnesium sulfate, filtered, and evaporated. The residue was then dissolved in tetrahydrofuran (10 ml) and added under nitrogen to a mixture of tris(dibenzylidineacetone)dipalladium (0) (11.3 mg, 0.012 mmol), racemic 2,2′-bis(diphenylphosphino)-1,1 ′-binaphthyl (21.7 mg, 0.035 mmol), sodium t-butoxide (193 mg, 2.01 mmol) in a dry tube. Benzophenone imine (0.34 ml, 367 mg, 2.02 mmol) was then added to the mixture and the tube was sealed under nitrogen. The solution was then stirred at 100° C. (bath temperature) overnight. The solution was then allowed to cool. and was evaporated. The residue was partitioned between brine and dichloromethane, and the organic layer was dried (magnesium sulfate), filtered, and evaporated. To the residue, sodium acetate trihydrate (717 mg, 5.26 mmol), hydroxylamine hydrochloride (292 mg, 4.20 mmol), and methanol (20 ml) were added, and the resulting solution was stirred overnight at room temperature. The solution was evaporated, the residue was partitioned between aqueous sodium hydroxide and chloroform, and the organic extract was dried (magnesium sulfate), filtered, and evaporated. To the residue, 2-thiophenecarboximidothioic acid ethyl ester hydrochloride (833 mg, 4.00 mmol), ethanol (20 ml), and diisopropylethylamine (0.69 ml) were added, and the solution was heated under nitrogen at 80° C. (bath temperature) overnight. The solution was evaporated, the residue was partitioned between aqueous sodium hydroxide and chloroform, and the organic extract was dried (magnesium sulfate), filtered, and evaporated. The residue was then dissolved in methanol (20 ml), and aqueous hydrochloric acid (1 ml) was added. After 30 min, the solution was evaporated. The product was purified by reverse phase HPLC on a Waters Bondapak® C₁₈ column using a gradient of acetonitrile and 0.1% aqueous trifluoroacetic acid as the eluent. The product-containing fractions were evaporated. The residue was dissolved in aqueous hydrochloric acid, and the solution was evaporated. The residue was co-evaporated with ethanol and then with t-butyl methyl ether to give the dihydrochloride salt of the title compound as a solid (52 mg, 0.14 mmol, 17%). MS (ES⁺) ^{m}/z 302 (100%, MH⁻).

### EXAMPLE 32

Alternative Preparation of N-[4-Methoxy-3-(1-methyl-2-pyrrolidinyl)phenyl]-2thiophenecarboximidamide

a) 1-(5-Bromo-2-methoxyphenyl)-4-chlorobutan-1-one

To a solution of 4-bromoanisole (25 g, 133.7 mmol) and 4-chlorobutanoyl chloride (24 mL, 213.92 mmol) in an Erlenmeyer flask was added aluminium chloride (26.7 g, 200.6 mmol) portionwise at 0° C. with occasional stirring. The resulting yellow-tan. viscous foam was allowed to warm to ambient temperature and left to stand for 45 min before being quenched by addition of ice (300 g). The resultant white slurry was basified with 2.0 N sodium hydroxide solution to pH 13 and extracted into diethyl ether (3×250 mL), wherein the combined extracts were washed with water (1×150 mL) and saturated brine (1×100 mL). Concentration by rotary evaporation yielded a yellow oil which was purified via flash chromatography (silica gel, gradient elution: diethyl ether/hexane=5:95 to 8:92 as an eluent) giving rise to the desired 1-(5-bromo-2-methoxyphenyl)-4-chlorobutan-1-one (21.47 g, 55%). Mass Spectrum (API+): M+1: ^{m}/z 291, 293, 295.

b) 2-(5-Bromo-2-methoxyphenyl)-1-methylpyrrolidine

To a stirring solution of 1-(5-bromo-2-methoxyphenyl)-4-chlorobutan-1-one (8.8 g, 30.18 mmol) in methanol (25 mL) was added methylamine (100 mL, 2 M in methanol). Upon complete addition, the flask was sealed and warmed to 35° C. for 4.5 h then cooled to 0° C. and powdered sodium borohydride (1.71 g, 45.27 mmol) was added in small portions over 5 min wherein the reaction was left to stir under a nitrogen purge, warming to ambient temperature. After 10 h the opaque yellow mixture was diluted with water (˜20 mL) and acidified to pH 1 by dropwise addition of 6N hydrochloric acid and concentrated in vacuo. The aqueous remains were extracted with diethyl ether (3×120 mL) then basified to pH 14 (initial addition of solid sodium bicarbonate, then 20% aqueous sodium hydroxide solution) and again extracted with diethyl ether (3×120 mL). The combined basic organic extracts were washed with saturated brine (1×100 mL), dried over sodium sulfate, filtered and concentrated by rotary evaporation to afford the viscous yellow oil, 2-(5-bromo-2-methoxyphenyl)-1-methylpyrrolidine (4.55 g, 56%). Mass Spectrum (API+): M+1: ^{m}/z 270, 272.

c) 4-Methoxy-3-(1-methylpyrrolidin-2-yl)phenylamine

To an oven dried Schlenk flask, charged with tris(dibenzylidineacetone)dipalladium(0) (44 mg, 0.048 mmol), (R)-(+)-2,2′-bis(diphenylphosphino)-1,1′-binaphthyl (89 mg, 0.143 mmol) and sodium t-butoxide (1.28 g, 13.31 mmol) under a nitrogen purge, was added via syringe, an anhydrous, degassed toluene (40 mL) solution of benzophenone imine (1.19 mL, 11.4 mmol) and 2-(5-bromo-2-methoxyphenyl)-1-methylpyrrolidine (2.57 g, 9.51 mmol—subject to a silica plug, eluting with ethyl acetate and drying in vacuo directly prior to use). The flask was sealed under nitrogen, and the stirring reaction was heated to 80° C. for 10 h. Upon cooling, the mixture was diluted with diethyl ether (200 mL), filtered through Celite and concentrated in vacuo, giving rise to the orange-red intermediate, benzhydrylidene-[4-methoxy-3-(1-methylpyrrolidin-2-yl)-phenyl] amine. The oil was taken up in tetrahydrofuran (40 mL), acidified to pH 1 with 2N hydrochloric lo acid and left to stir for 45 min at ambient temperature before partitioning between diethyl ether and 0.5N hydrochloric acid. After being extracted with diethyl ether (3×40 mL), the aqueous remains were then basified to pH 12 with 20% aqueous sodium hydroxide and extracted with methylene chloride (3×40 mL). The combined methylene chloride extracts were washed with water (1×20 mL), dried over sodium sulfate, filtered and concentrated in vacuo, affording the desired product, 4-methoxy-3-(1-methylpyrrolidin-2-yl)phenylamine (˜2 g, crude-quantitative). Mass Spectrum (API+): M+1: 207 (100%).

d) N-[4-Methoxy-3(1-methyl-2-pyrrolidinyl)phenyl]-2-thiophenecarboximidamide Dihydrochloride Salt

A round-bottomed flask was charged with 4-methoxy-3-(1-methylpyrrolidin-2-yl)phenylamine (˜2 g, ˜9.5 mmol), 2-thiophenecarboximidothioic acid ethyl ester hydrochloride (2.74 g, 13.3 mmol) and absolute ethanol (20 mL). The stirring reddish solution was heated to 60° C. under a nitrogen atmosphere for 2.5 h then concentrated down by rotary evaporation. The resulting residue was adsorbed onto 3 times the mass of silica gel and subjected to flash chromatography (silica gel, gradient elution: 2.0M ammoniated methanol/methylene chloride 1:99 to 5:95 as an eluent) yielding a pale yellow residue which was then subjected to treatment with decolourising carbon. After filtration through Celite and concentration, the yellow residue was taken up in methylene chloride and converted into the dihydrochloride salt by the dropwise addition of 1N hydrochloric acid in diethyl ether. Concentration, followed by trituration with methanol and diethyl ether. filtration and drying in vacuo at 58° C./240 mTorr afforded the title compound N-[4-methoxy-3-(1-methylpyrrolidin-2-yl)phenyl]-2-thiophenecarboxamidine dihydrochloride (2.453 g, 66%). Mass Spectrum (API+): M+1: 316 (100%).

1. A compound of formula (I) 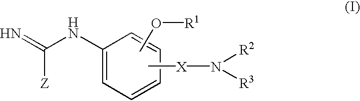
wherein
Z represents a furan or thiophene ring, optionally substituted by one or more substituents selected from halogen, trifluoromethyl, C1 to 6 alkyl, C1 to 6 alkoxy, hydroxy, amino, S(O)_{q}R⁴, CO₂R⁵ and CONR⁶R⁷;
X represents C1 to 6 alkyl;
R¹ represents hydrogen, C1 to 6 alkyl, C1 to 6 alkyl-O—R⁸, C1 to 6 alkyl-NR⁹R¹⁰ or phenyl;
said phenyl being optionally substituted by one or more substituents selected from halogen, trifluoromethyl, C1 to 6 alkyl, C1 to 6 alkoxy, hydroxy and amino;
R² and R³ independently represent hydrogen, C1 to 6 alkyl, C2 to 7 alkanoyl or —(CH₂)ₙ₋₁CH₃₋ₘFₘ;
or the group NR²R³ represents azetidinyl, pyrrolidinyl, piperidinyl, morpholinyl; or piperazinyl optionally 4-substituted by C1 to 6 alkyl;
or the groups X and R² are joined together such that the group X—N—R² represents a saturated 4 to 7 membered azacyclic ring;
R⁴, R⁵, R⁶ and R⁷ independently represent hydrogen or C1 to 6 alkyl;
R⁸ represents hydrogen, C1 to 6 alkyl or C1 to 6 alkyl substituted by hydroxy or C1 to 6 alkoxy;
R⁹ and R¹⁰ independently represent hydrogen or C1 to 6 alkyl;
or the group NR⁹R¹⁰ represents azetidinyl, pyrrolidinyl, piperidinyl, morpholinyl; or piperazinyl optionally 4-substituted by C1 to 6 alkyl;
m represents an integer 1, 2 or 3;
n represents an integer 1 to 6;
q represents an integer 0, 1 or 2;
and optical isomers, racemates and tautomers thereof and pharmaceutically acceptable salts thereof

2. A compound of formula (I), according to claim 1, wherein the substituent OR¹ in formula (I) is in the ortho or para position relative to the amidine group.

3. A compound of formula (I), according to claim 1, wherein the substituent OR¹ in formula (I) is in the para position relative to the amidine group.

4. A compound of formula (I), according to any one of claims 1 to 3, wherein the substituent —X—NR²R³ in formula (I) is in the meta position relative to the amidine group.

5. A compound of formula (I), according to any one of claims 1 to 4, wherein the group OR¹ represents methoxy or cyclopentyloxy.

6. A compound of formula (I), according to any one of claims 1 to 5, wherein X represents CH₂.

7. A compound of formula (I), according to claim 1, which is:
N-{4-methoxy-3-[(methylamino)methyl]phenyl}-2-thiophenecarboximidamide;
N-[4-methoxy-3-(1-pyrrolidinylmethyl)phenyl}-2-thiophenecarboximidamide;
N-[4-methoxy-3-(1)morpholinylmethyl)phenyl}-2-thiophenecarboximidamide;
N-{4-methoxy-3-[(4-methyl-1-piperazinyl)methyl[phenyl}-2-thiophenecarboximidamide;
N-{4-propoxy-3-[(methylamino)methyl[phenyl}-2-thiophenecarboximidamide;
N-{4-(cyclopentyloxy)-3-[(methylamino)methyl]phenyl}-2-thiophenecarboximidamide;
N-[4-cyclopentyloxy-3-(1-pyrrolidinylmethyl)phenyl}-2-thiophenecarboximidamide;
N-[4-methoxy-3-(1-morpholinylmethyl)phenyl}-3-thiophenecarboximidamide;
N-{2-methoxy-5-[(methylamino)methyl]phenyl}-2-thiophenecarboximidamide;
N-{2-methoxy-3-[(methylamino)methyl]phenyl}-2-thiophenecarboximidamide;
N-{4-methoxy-3-[(methylamino)methyl]phenyl}-2-furancarboximidamide;
N-(3-{[(2-fluoroethyl)amino]methyl}-4-methoxyphenyl)-2-thiophenecarboximidamide;
N-[3-(aminomethyl)-4-methoxyphenyl)-2-thiophenecarboximidamide;
N-(3-{[(2,2-difluoroethyl)amino)methyl-4-methoxyphenyl)-2-thiophene-carboximidamide;
N-(4-methoxy-3-{[(2,2,2-trifluoroethyl)amino]methyl}phenyl)-2-thiophenecarboximidamide;
N-(3-[(cyclopropylamino}methyl]-4-methoxyphenyl}-2-thiophenecarboximidamide;
N-{3-[(diethylamino)methyl]-4-methoxyphenyl}-2-thiophenecarboximidamide;
N-{3-[(isopropylamino)methyl]-4-methoxyphenyl}-2-thiophenecarboximidamide;
N-{4-isopropoxy-3-[{methylamino}methyl]phenyl}-2-thiophenecarboximidamide;
N-{4-isopropoxy-3-[{isopropylamino}methyl]phenyl}-2-thiophenecarboximidamide;
N-[4-isopropoxy-3-(1-pyrrolidinylmethyl)phenyl]-2-thiophenecarboximidamide;
N-{4-(1-ethylpropoxy)-3-[(methylamino)methyl]phenyl}-2-thiophenecarboximidamide;
N-{4-(1-ethylpropoxy)-3-[(isopropylamino)methyl]phenyl}-2-thiophenecarboximidamide;
N-{4-cyclopentyloxy-3-[(isopropylamino)methyl]phenyl]-2-thiophenecarboximidamide;
N-{3-[(methylamino)methyl]-4-phenoxyphenyl}-2-thiophenecarboximidamide;
N-{3-[(isopropylamino)methyl]-4-phenoxyphenyl}-2-thiophenecarboximidamide;
N-{3-[(cyclopropylamino)methyl]-4-phenoxyphenyl}-2-thiophenecarboximidamide;
N-(3-{[(2-fluoroethyl)amino]methyl}-4-phenoxyphenyl)-2-thiophenecarboximidamide;
N-{4-methoxy-3-[1-(methylamino)ethyl]phenyl}-2-thiophenecarboximidamide;
N-[4-methoxy-3-(1-methyl-2-pyrrolidinyl)phenyl]-2-thiophenecarboximidamide;
N-[4-methoxy-3-(2-pyrrolidinyl)phenyl]-2-thiophenecarboximidamide;
or an optical isomer, racemate or tautomer of any one thereof or a pharmaceutically acceptable salt of any one thereof.

8. A compound of formula (I), as defined in any one of claims 1 to 7, for use as a medicament.

9. A pharmaceutical formulation comprising a compound of formula (I), as defined in any one of claims 1 to 7, or an optical isomer, racemate or tautomer thereof or a pharmaceutically acceptable salt thereof, optionally in admixture with a pharmaceutically acceptable diluent or carrier.

10. A method of treating, or reducing the risk of, a human disease or condition in which inhibition of nitric oxide synthase activity is beneficial which comprises administering to a person suffering from or susceptible to such a disease or condition, a therapeutically effective amount of a compound of formula (I), as defined in any one of claims 1 to 7, or an optical isomer, racemate or tautomer thereof or a pharmaceutically acceptable salt thereof.

11. A method of treatment according to claim 10 in which it is predominantly the neuronal isoform of nitric oxide synthase that is inhibited.

12. A method of treating, or reducing the risk of hypoxia or stroke or ischaemia or neurodegenerative conditions or schizophrenia or anxiety or pain or migraine, which comprises administering to a person suffering from or susceptible to such a disease or condition a therapeutically effective amount of a compound of formula (I), as defined in any one of claims 1 to 7, or an optical isomer, racemate or tautomer thereof or a pharmaceutically acceptable salt thereof.

13. A method of treatment according to claim 12, wherein the condition to be treated is selected from the group consisting of hypoxia, ischaemia, stroke, Parkinson's disease, anxiety, schizophrenia, migraine and pain.

14. A method of treatment according to claim 13, wherein the condition to be treated is stroke.

15. A method of treatment according to claim 13, wherein the condition to be treated is pain.

16. A method of treatment according to claim 13, wherein the condition to be treated is migraine

17. A method of treatment according to claim 13, wherein the condition to be treated is schizophrenia.

18. A method of treatment according to claim 13, wherein the condition to be treated is Parkinson's disease

19. The use of a compound of formula (I) as defined in any one of claims 1 to 7, or an optical isomer, racemate or tautomer thereof or a pharmaceutically acceptable salt thereof, in the manufacture of a medicament for the treatment or prophylaxis of human diseases or conditions in which inhibition of nitric oxide synthase activity is beneficial.

20. The use as claimed in claim 19 wherein it is predominantly the neuronal isoform of nitric oxide synthase that is inhibited.

21. The use of a compound of formula (I) as defined in any one of claims 1 to 7, or an optical isomer, racemate or tautomer thereof or a pharmaceutically acceptable salt thereof, in the manufacture of a medicament for the treatment or prophylaxis of hypoxia or stroke or ischaemia or neurodegenerative conditions or schizophrenia or anxiety or pain or migraine.

22. The use as claimed in claim 21, wherein the condition is selected from the group consisting of hypoxia, ischaemia, stroke, Parkinson's disease, anxiety, schizophrenia, migraine and pain.

23. The use as claimed in claim 22, wherein the condition is stroke.

24. The use as claimed in claim 22, wherein the condition is pain.

25. The use as claimed in claim 22, wherein the condition is migraine.

26. The use as claimed in claim 22, wherein the condition is schizophrenia.

27. The use as claimed in claim 22, wherein the condition is Parkinson's disease.

28. A process for the preparation of a compound of formula (I), as defined in any one of claims 1 to 7, and optical isomers, racemates and tautomers thereof and pharmaceutically salts thereof, which comprises preparing a compound of formula (I) by:
(a) reacting a corresponding compound of formula (II) or a salt thereof 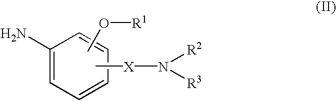
wherein R¹, R², R³ and X are as defined in claim 1, with a compound of formula (III) or a salt thereof 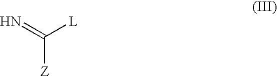
wherein Z is as defined in claim 1 and L represents a leaving group; or
(b) reacting a corresponding compound of formula (IV) or a salt thereof 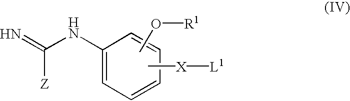
wherein R¹, X and Z are as defined in claim 1 and L¹ is a leaving group, with a compound of formula HNR²R³ or a salt thereof, wherein R² and R³ are as defined in claim 1; or
(c) preparing a compound of formula (I) wherein X represents —CH₂— by reduction of a corresponding compound wherein X represents —CO— (formula V) 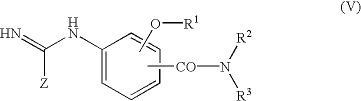
and where desired or necessary converting the resultant compound of formula (I), or another salt thereof, into a pharmaceutically acceptable salt thereof, or vice versa, and where desired converting the resultant compound of formula (I) into an optical isomer thereof.

29. A intermediate compound of formula (V) 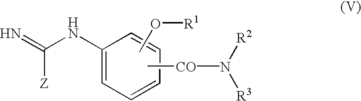
wherein R¹, R², R³ and Z are as defined in claim 1.
